(19) United States
(12) Patent Application Publication
(10) Pub. No.: US 2013/0317064 A1
(43) Pub. Date: Nov. 28, 2013

(54) MICROBIOCIDAL PYRAZOLE DERIVATIVES

(71) Applicants: Clemens Lamberth, Stein (CH); Sarah Sulzer-Mosse, Stein (CH); Fredrik Cederbaum, Stein (CH); Guillaume Berthon, Stein (CH)

(72) Inventors: Clemens Lamberth, Stein (CH); Sarah Sulzer-Mosse, Stein (CH); Fredrik Cederbaum, Stein (CH); Guillaume Berthon, Stein (CH)

(73) Assignee: SYNGENTA PARTICIPATIONS AG, Basel (CH)

(21) Appl. No.: 13/984,459

(22) PCT Filed: Feb. 8, 2012
(86) PCT No.: PCT/EP12/52110

§ 371 Date: Aug. 8, 2013

(30) Foreign Application Priority Data
Feb. 10, 2011 (EP) 11153986.2
Jul. 4, 2011 (EP) 11172561.0

(51) Int. Cl. A01N 43/78; C07D 417/14

(52) U.S. Cl. CPC A01N 43/78; C07D 417/14
USPC 514/326; 546/209

(57) ABSTRACT

The present invention provides compounds of formula I: wherein the substituents are as defined in claim 1, are useful as active ingredients, which have microbiocidal activity, in particular fungicidal activity.

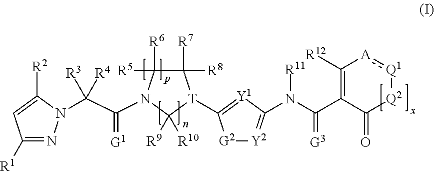

The present invention relates to microbiocidal pyrazole derivatives, e.g. as active ingredients, which have microbiocidal activity, in particular fungicidal activity. The invention also relates to preparation of these pyrazole derivatives, to pyrazole derivatives used as intermediates in the preparation of these pyrazole derivatives, to preparation of these intermediates, to agrochemical compositions which comprise at least one of the pyrazole derivatives, to preparation of these compositions and to use of the pyrazole derivatives or compositions in agriculture or horticulture for controlling or preventing infestation of plants, harvested food crops, seeds or non-living materials by phytopathogenic microorganisms, preferably fungi.

Certain compounds for use as fungicides are described in WO 2007/014290, WO 2008/013622, WO 2008/013925, WO 2008/091580, WO 2008/091594 and WO 2009/055514.

The present invention provides compounds of formula I:

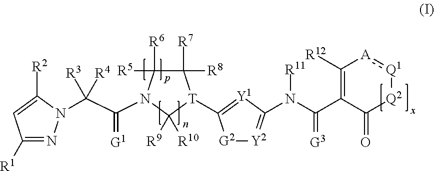

wherein
G¹, G² and G³ are independently O or S;

### T is CR¹³ or N;

Y¹ and Y² are independently CR¹⁴ or N;

### A is C(R¹⁵R¹⁶), C(═O), C(═S), NR²¹, O or S;

Q¹ is C(R¹⁷R¹⁸), C(═O), C(═S), NR²¹, O or S; Q² is C(R¹⁹R²⁰), C(═O), C(═S), NR²¹, O or S;
the bond between A and Q¹ is a single bond or a double bond;
n is 1 or 2;
p is 1 or 2, providing that when n is 2, p is 1;
x is 0 or 1, providing that when x is 1, Q¹ and Q² cannot both be oxygen;
R¹, R², R³, R⁴, R⁵, R⁶, R⁷, R⁸, R⁹, R¹⁰, R¹³ and R¹⁴ each independently are hydrogen, halogen, cyano, C₁-C₄alkyl, C₃-C₅cycloalkyl or C₁-C₄haloalkyl;
R¹¹ is hydrogen, C₁-C₄alkyl, C₃-C₅cycloalkyl or C₁-C₄alkoxy;
R¹² is hydroxyl, O⁻M⁺, OC(═O)R²⁵, amino or NHR²²;
M⁺ is a metal cation or ammonium cation,
R¹⁵, R¹⁶, R¹⁷, R¹⁸, R¹⁹ and R²⁰ each independently are hydrogen, halogen, hydroxyl, amino, cyano, C₁-C₈alkyl, C₁-C₈alkylcarbonyl, C₂-C₈alkenyl, C₂-C₈alkynyl, C₃-C₈cycloalkyl, C₁-C₈alkoxy, C₁-C₈alkoxycarbonyl, C₁-C₈alkylthio, C₁-C₈alkylsulfonyl, C₁-C₈alkylsulfinyl, aryl, arylcarbonyl, heteroaryl or NHR²², wherein the alkyl, alkenyl, alkynyl, cycloalkyl, alkoxy, aryl and heteroaryl are optionally substituted by one or more R²³; and wherein
R¹⁵ and R¹⁶, R¹⁷ and R¹⁸, and/or R¹⁹ and R²⁰ may together form a saturated three- to six-membered alicyclic or heterocyclic ring wherein the aliyclic and heterocyclic rings are optionally substituted by one or more R²⁴; and/or
R¹⁵ and R¹⁷, and/or R¹⁸ and R¹⁹ may together form a saturated or partially unsaturated four- to seven-membered alicyclic or heterocyclic ring wherein the aliyclic and heterocyclic rings are optionally substituted by one or more R²⁴; and/or
R¹⁵ and R¹⁹ may together form a saturated or partially unsaturated four- to seven-membered alicyclic or heterocyclic ring wherein the aliyclic and heterocyclic rings are optionally substituted by one or more R²⁴;
R²¹ and R²² each independently are hydrogen, C₁-C₈alkyl, C₁-C₈haloalkyl C₂-C₈alkenyl, C₁-C₈haloalkenyl C₂-C₈alkynyl, C₂-C₈haloalkynyl, C₃-C₈cycloalkyl, C₃-C₈halocycloalkyl, C₁-C₈alkoxy, C₁-C₈haloalkoxy, C₁-C₈alkylcarbonyl, C₁-C₈alkoxycarbonyl, C₁-C₈haloalkylcarbonyl, C₁-C₈alkylsulfonyl, C₁-C₈haloalkylsulfonyl, amino, NH(C₁-C₈alkyl), N(C₁-C₈alkyl)₂, aryl or heterocyclyl, wherein aryl and heterocyclyl are optionally substituted by one or more R²⁴;
each R²³ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkyloxy, C₃-C₈cycloalkyl-C₁-C₄alkylthio, C₁-C₈alkoxy, C₃-C₈cycloalkyloxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkyloxy, aryl-C₁-C₄alkylthio, heterocyclyl, heterocycyl-C₁-C₄alkyl, heterocycyl-C₁-C₄alkyloxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R²⁴;
each R²⁴ independently is halogen, cyano, C₁-C₄alkyl, C₁-C₄haloalkyl, C₁-C₄alkoxy or C₁-C₄haloalkoxy; and
R²⁵ is C₁-C₆alkyl or C₁-C₆alkoxy; or a salt or a N-oxide thereof.

Where substituents are indicated as being optionally substituted, this means that they may or may not carry one or more identical or different substituents, e.g. one to five substituents, e.g. one to three substituents. Normally not more than three such optional substituents are present at the same time. Where a group is indicated as being substituted, e.g. alkyl, unless stated otherwise this includes those groups that are part of other groups, e.g. the alkyl in alkylthio.

The term “halogen” refers to fluorine, chlorine, bromine or iodine, preferably fluorine, chlorine or bromine.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl, iso-amyl or pivaloyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Haloalkenyl groups are alkenyl groups, respectively, which are substituted with one or more of the same or different halogen atoms and are, for example, 2,2-difluorovinyl or 1,2-dichloro-2-fluoro-vinyl.

Haloalkynyl groups are alkynyl groups, respectively, which are substituted with one or more of the same or different halogen atoms and are, for example, 1-chloro-prop-2-ynyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a —CN group.

Amino means an NH₂ group.

Hydroxyl or hydroxy stands for a —OH group.

Aryl means a ring system which may be mono-, bi- or tricyclic. Examples of such rings include phenyl, naphthalenyl, anthracenyl, indenyl or phenanthrenyl. A preferred aryl group is phenyl.

Heteroaryl stands for aromatic ring systems comprising mono-, bi- or tricyclic systems wherein at least one oxygen, nitrogen or sulfur atom is present as a ring member. Monocyclic and bicyclic aromatic ring systems are preferred, monocyclic ring systems are more preferred. For example, monocyclic heteoraryl may be a 5- to 7-membered aromatic ring containing one to three heteroatoms selected from oxygen, nitrogen and sulfur, more preferably selected from nitrogen and sulfur. Bicyclic heteroaryl may be a 9- to 11-membered bicyclic ring containing one to five heteroatoms, preferably one to three heteroatoms, selected from oxygen, nitrogen and sulfur. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, imiazothiazoyl, quinolinyl, quinoxalinyl, isoquinolinyl, phthalazinyl, quinoxalinyl, quinazolinyl, cinnolinyl and naphthyridinyl, preferably pyridyl, pyrazinyl, pyridazinyl, pyrimidinyl, pyrrolyl, pyrazolyl, imidazolyl, triazolyl, furanyl, thienyl thiazolyl or thiadiazolyl. Heteroaryl rings do not contain adjacent oxygen ring atoms, adjacent sulfur ring atoms or adjacent oxygen and sulfur ring atoms. A link to a heteroaryl group can be via a carbon atom or via a nitrogen atom.

Heterocyclyl is defined to include heteroaryl and in addition their unsaturated or partially unsaturated analogues.

The compounds of formula I may occur in different tautomeric forms, for example, if R¹² is hydroxyl, in the formulas I.a, I.b and I.c. Each form is included within the compounds of formula I.

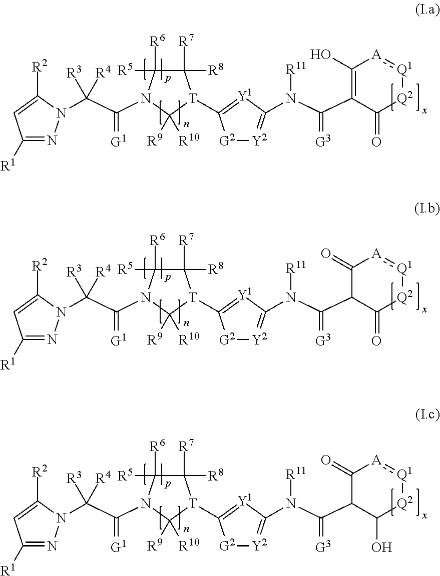

The presence of one or more possible asymmetric carbon atoms in a compound of formula I means that the compounds may occur in optically isomeric forms, i.e. enantiomeric or diastereomeric forms. Also atropisomers may occur as a result of restricted rotation about a single bond. Formula I is intended to include all those possible isomeric forms and mixtures thereof. The present invention includes all those possible isomeric forms and mixtures thereof for a compound of formula I. Likewise, formula I is intended to include all possible tautomers. The present invention includes all possible tautomeric forms for a compound of formula I.

In each case, the compounds of formula I according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents G¹, G², G³, T, Y¹, Y², A, Q¹, Q², n, p, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ and R²⁵ with reference to compounds of formula I and other compounds of the invention carrying the same substituents. For any one of these substituents, any of the definitions given below may be combined with any definition of any other substituent given below or elsewhere in this document.

G¹, G² and G³ are independently O or S.

G¹ is preferably O.

G² is preferably S.

G³ is preferably O.

T is CR¹³ or N, preferably CH or N, more preferably CH.

Y¹ and Y² are independently CR¹⁴ or N.

Y¹ is preferably CH or N, more preferably N.

Y² is preferably CH or N; more preferably CH.

n is 1 or 2, preferably 2.

p is 1 or 2, providing that when n is 2, p is 1, preferably p is 1.

x is 1 or 0, preferably 1.

A is C(R¹⁵R¹⁶), C(═O), C(═S), NR²¹, O or S; Q¹ is C(R¹⁷R¹⁸), C(═O), C(═S), NR²¹, O or S; Q² is C(R¹⁹R²⁰), C(═O), C(═S), NR²¹, O or S. Preferably there are no —O—O—, —S—S—, —O—S— or —S—O— in the ring formed by A, Q¹ and Q². Preferably there are no adjacent C═O groups in the ring formed by A, Q¹ and Q². Preferably no more than two of A, Q¹ and Q² are NR²¹, O or S. Preferably the bond between A and Q¹ is a single bond or a double bond;

In one group of compounds there are no adjacent heteroatoms in the ring formed by A, Q¹ and Q². In another group of compounds no more than one of A, Q¹ and Q² are NR²¹, O or S. In another group of compounds when x is 1 A is C(R¹⁵R¹⁶), NR¹⁷, O or S; Q¹ is C(R¹⁷R¹⁸), C(═O), C(═S), NR¹⁷, O or S; and Q² is C(R¹⁹R²⁰), NR¹⁷, O or S. In another group of compounds when x is 0, A is C(R¹⁵R¹⁶), C(═O), C(═S), NR¹⁷, O or S; Q¹ is C(R¹⁷R¹⁸), NR¹⁷, O or S. Even more preferred options for A, Q¹ and Q² are depicted by Z1 to Z19 in formula I.d (see below).

R¹, R², R³, R⁴, R⁵, R⁶, R⁷, R⁸, R⁹, R¹⁰, R¹³ and R¹⁴ each independently are hydrogen, halogen, cyano, C₁-C₄alkyl or C₁-C₄haloalkyl, preferably hydrogen, halogen, C₁-C₄alkyl or C₁-C₄haloalkyl.

Preferably R¹ and R² are each independently halogen, methyl or halomethyl, more preferably methyl or halomethyl, more preferably methyl or trifluoromethyl. Preferably R¹ is trifluoromethyl. Preferably R² is methyl. In one group of compounds R¹ is trifluoromethyl and R² is methyl. In another group of compounds R¹ and R² are both difluoromethyl.

Preferably R³, R⁴, R⁵, R⁶, R⁷, R⁸, R⁹, R¹⁰, R¹³ and R¹⁴ are each independently hydrogen, halogen, C₁-C₄alkyl or C₁-C₄haloalkyl, more preferably hydrogen, halogen, methyl or halomethyl, even more preferably hydrogen or methyl, most preferably hydrogen.

R¹¹ is hydrogen, C₁-C₄alkyl, C₃-C₅cycloalkyl or C₁-C₄alkoxy; preferably hydrogen, C₁-C₄alkyl or C₁-C₄alkoxy, more preferably hydrogen or methyl, even more preferably hydrogen.

R¹² is hydroxyl, O⁻M⁺, OC(═O)R²⁵, amino or NHR²²; preferably hydroxyl, O⁻M⁺, or NHR²², more preferably hydroxyl or O⁻M⁺, even more preferably hydroxyl.

M⁺ is a metal cation or ammonium cation, preferably a metal cation, e.g. an alkali metal cation, such as potassium, sodium or lithium.

R¹⁵, R¹⁶R¹⁷, R¹⁸, R¹⁹ and R²⁰ each independently are hydrogen, halogen, hydroxyl, amino, cyano, C₁-C₈alkyl, C₁-C₈alkylcarbonyl, C₂-C₈alkenyl, C₂-C₈alkynyl, C₃-C₈cycloalkyl, C₁-C₈alkoxy, C₁-C₈alkoxycarbonyl, C₁-C₈alkylthio, C₁-C₈alkylsulfonyl, C₁-C₈alkylsulfinyl, aryl, arylcarbonyl, heteroaryl or NHR²², wherein alkyl, alkenyl, alkynyl, cycloalkyl, alkoxy, aryl and heteroaryl are optionally substituted by one or more R²³.

Preferably R¹⁵, R¹⁶R¹⁷, R¹⁸, R¹⁹ and R²⁰ each independently are hydrogen, halogen, hydroxyl, cyano, C₁-C₈alkyl, C₂-C₈alkenyl, C₂-C₈alkynyl, C₃-C₈cycloalkyl, C₁-C₈alkoxy, C₁-C₈alkoxycarbonyl, C₁-C₈alkylthio, aryl, arylcarbonyl, heteroaryl or NHR²², wherein alkyl, alkenyl, alkynyl, cycloalkyl, alkoxy, aryl and heteroaryl are optionally substituted by one or more R²³.

Preferably R¹⁵, R¹⁶, R¹⁷, R¹⁸, R¹⁹ and R²⁰ each independently are hydrogen, halogen, hydroxyl, cyano, C₁-C₈alkyl, C₂-C₈alkenyl, C₂-C₈alkynyl, C₃-C₈cycloalkyl, C₁-C₈alkoxy, C₁-C₈alkoxycarbonyl, C₁-C₈alkylthio, aryl, heteroaryl or NHR²², wherein alkyl, alkenyl, alkynyl, cycloalkyl, alkoxy, aryl and heteroaryl are optionally substituted by one or more R²³ and wherein each heteroaryl is independently selected from pyridyl, pyrazinyl, pyridazinyl, pyrimidinyl, pyrrolyl, pyrazolyl, imidazolyl, triazolyl, furanyl, thienyl thiazolyl and thiadiazolyl.

Even more preferably R¹⁵, R¹⁶R¹⁷, R¹⁸, R¹⁹ and R²⁰ each independently are hydrogen, halogen, cyano, C₁-C₄alkyl, C₃-C₆cycloalkyl-C₁-C₄alkyl, C₃-C₆cycloalkyl-C₁-C₄alkyl wherein one ring atom is replaced by oxygen, C₂-C₄alkenyl, C₂-C₄alkynyl, C₃-C₆cycloalkyl, C₁-C₄alkylthio-C₃-C₆cycloalkyl, phenylthio-C₃-C₆cycloalkyl, benzylthio-C₃-C₆cycloalkyl, C₃-C₆cycloalkyl wherein one ring atom is replaced by oxygen, C₁-C₄alkoxy, C₁-C₄alkylthio, C₁-C₄alkylcarbonylamino, wherein alkyl, alkenyl, alkynyl, and cycloalkyl are optionally substituted by one to five halogen, and wherein phenyl and benzyl are optionally substituted by one to five groups selected from halogen, cyano, C₁-C₄alkyl, C₁-C₄haloalkyl, C₁-C₄alkoxy and C₁-C₄haloalkoxy.

R¹⁵ and R¹⁶, R¹⁷ and R¹⁸, and/or R¹⁹ and R²⁰ may together form a saturated three- to six-membered alicyclic or heterocyclic ring wherein the aliyclic and heterocyclic rings are optionally substituted by one or more R²⁴; and/or

R¹⁵ and R¹⁷, and/or R¹⁸ and R¹⁹ may together form a saturated or partially unsaturated four- to seven-membered alicyclic or heterocyclic ring wherein the aliyclic and heterocyclic rings are optionally substituted by one or more R²⁴; and/or

R¹⁵ and R¹⁹ may together form a saturated or partially unsaturated four- to seven-membered alicyclic or heterocyclic ring wherein the aliyclic and heterocyclic rings are optionally substituted by one or more R²⁴.

A heterocyclic ring formed by any of R¹⁵ and R¹⁶, R¹⁷ and R¹⁸, R¹⁹ and R²⁰, R¹⁵ and R¹⁷, R¹⁸ and R¹⁹, and R¹⁵ and R¹⁹ contains for example one to three heteroatoms selected from O, S, and N(R²⁴).

Preferably R¹⁵ and R¹⁶, R¹⁷ and R¹⁸, and/or R¹⁹ and R²⁰ may together form a saturated three- to six-membered alicyclic or heterocyclic ring wherein the aliyclic and heterocyclic rings are optionally substituted by one or more R²⁴; and/or

R¹⁵ and R¹⁷ may together form a saturated or partially unsaturated four- to seven-membered alicyclic or heterocyclic ring wherein the aliyclic and heterocyclic rings are optionally substituted by one or more R²⁴; and/or

R¹⁵ and R¹⁹ may together form a saturated or partially unsaturated four- to seven-membered alicyclic or heterocyclic ring wherein the aliyclic and heterocyclic rings are optionally substituted by one or more R²⁴; and where said heterocyclic rings preferably contain one or two heteroatoms selected from O, S and NR²⁴.

More preferably one or two of the pairs R¹⁵ and R¹⁶, R¹⁷ and R¹⁸, and R¹⁹ and R²⁰ may together form a saturated three- to six-membered alicyclic or heterocyclic ring wherein the aliyclic and heterocyclic rings are optionally substituted by one or more R²⁴; or

R¹⁵ and R¹⁷ may together form a saturated or partially unsaturated four- to seven-membered alicyclic or heterocyclic ring wherein the aliyclic and heterocyclic rings are optionally substituted by one or more R²⁴; or

R¹⁵ and R¹⁹ may together form a saturated or partially unsaturated four- to seven-membered alicyclic or heterocyclic ring wherein the aliyclic and heterocyclic rings are optionally substituted by one or more R²⁴; and where said heterocyclic rings preferably contain one heteroatom selected from O, S and NR²⁴.

Even more preferably one or two of the pairs R¹⁵ and R¹⁶, R¹⁷ and R¹⁸, and R¹⁹ and R²⁰ may together form a saturated three- to six-membered alicyclic ring wherein one of the ring members is optionally replaced by O, S, NH(C₁-C₄alkyl), NH(C₁-C₄alkoxy), and wherein the alicyclic ring is optionally substituted by one to five groups selected from halogen, methyl and halomethyl; or

R¹⁵ and R¹⁷ may together form a saturated four- to seven-membered alicyclic ring optionally substituted by one to five groups independently selected from halogen, methyl and halomethyl; or

R¹⁵ and R¹⁹ may together form a saturated four- to seven-membered alicyclic ring optionally substituted by one to five groups independently selected from halogen, methyl and halomethyl.

Each R²¹ and R²² independently are hydrogen, C₁-C₈alkyl, C₁-C₈haloalkyl C₂-C₈alkenyl, C₂-C₈haloalkenyl, C₂-C₈alkynyl, C₂-C₈haloalkynyl, C₃-C₈cycloalkyl, C₃-C₈halocycloalkyl, C₁-C₈alkoxy, C₁-C₈haloalkoxy, C₁-C₈alkylcarbonyl, C₁-C₈alkoxycarbonyl, C₁-C₈haloalkylcarbonyl, C₁-C₈alkylsulfonyl or C₁-C₈haloalkylsulfonyl, amino, NH(C₁-C₈alkyl), N(C₁-C₈alkyl)₂, aryl or heterocycyl, wherein aryl and heterocyclyl are optionally substituted by one or more R²⁴.

Preferably each R²¹ and R²² independently are hydrogen, C₁-C₈alkyl, C₁-C₈haloalkyl, C₂-C₈alkenyl, C₂-C₈haloalkenyl, C₂-C₈alkynyl, C₂-C₈haloalkynyl, C₃-C₈cycloalkyl, C₃-C₈halocycloalkyl, C₁-C₈alkoxy, C₁-C₈haloalkoxy, C₁-C₈alkylcarbonyl, C₁-C₈alkoxycarbonyl, C₁-C₈haloalkylcarbonyl, C₁-C₈alkylsulfonyl or C₁-C₈haloalkylsulfonyl, amino, NH(C₁-C₈alkyl), N(C₁-C₈alkyl)₂, phenyl or heterocycyl, wherein phenyl and heterocyclyl are optionally substituted by one or more R²⁴ and wherein each heterocycle is independently selected from pyrrolidinyl, pryollyl, imidazolyl, triazolyl, piperazinyl, piperidinyl, morpholinyl, pyridyl, pyrazinyl, pyridazinyl and pyrimidinyl.

More preferably each R²¹ and R²² independently are hydrogen, C₁-C₈alkyl, C₂-C₈alkenyl, C₂-C₈alkynyl, C₃-C₈cycloalkyl, C₁-C₈alkoxy, C₁-C₈alkylcarbonyl, NH(C₁-C₈alkyl), N(C₁-C₈alkyl)₂, phenyl, or a group selected from B1-B4

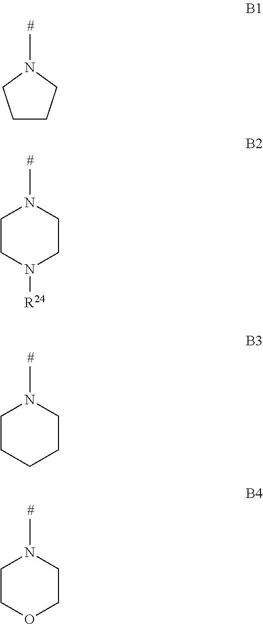

wherein the phenyl and B1-B4 are optionally substituted by one or more R²⁴.

Even more preferably each R²¹ and R²² independently are hydrogen, C₁-C₄alkyl, C₃-C₆cycloalkyl, C₁-C₄alkoxy, NH(C₁-C₄alkyl), N(C₁-C₄alkyl)₂, phenyl, B1 or B3, wherein phenyl and groups B1 and B3 are optionally substituted by one to five groups independently selected from halogen, methyl and halomethyl.

Each R²³ is independently, halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkyloxy, C₃-C₈cycloalkyl-C₁-C₄alkylthio, C₁-C₈alkoxy, C₃-C₈cycloalkyloxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkyloxy, aryl-C₁-C₄alkylthio, heterocyclyl, heterocycyl-C₁-C₄alkyl, heterocycyl-C₁-C₄alkyloxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein the aryl and heterocyclyl are optionally substituted by one or more R²⁴.

Preferably each R²³ independently is halogen, cyano, amino, nitro, hydroxyl, mercapto, C₁-C₈ alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkylthio, C₁-C₈alkoxy, C₃-C₈cycloalkyloxy, C₂-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, phenyl, phenyloxy, phenylthio, phenylsulfonyl, phenylsulfinyl, phenyl-C₁-C₄alkyl, phenyl-C₁-C₄alkyloxy, phenyl-C₁-C₄alkylthio, heterocyclyl, heterocycyl-C₁-C₄alkyl, heterocycyl-C₁-C₄alkyloxy, heterocycyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R²⁴; and wherein heterocyclyl is independently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydropyranyl.

More preferably each R²³ independently is halogen, cyano, amino, mercapto, C₁-C₈alkyl, C₃-C₈cycloalkyl, C₃-C₈cycloalkyl-C₁-C₄alkyloxy, C₃-C₈cycloalkyl-C₁-C₄alkylthio, C₁-C₈alkoxy, C₁-C₈alkylthio, phenyl, phenyloxy, phenylthio, phenyl-C₁-C₄alkoxy, phenyl-C₁-C₄alkylthio, heterocyclyl, heterocyclyl-C₁-C₄alkoxy, heterocyclyl-C₁-C₄alkylthio, NH(C₁-C₈alkyl), N(C₁-C₈alkyl)₂, and wherein heterocyclyl is independently selected from pyridyl, pyrazinyl, pyridazinyl, pyrimidinyl, pyrrolyl, pyrazolyl, imidazolyl, triazolyl, furanyl, thienyl, thiazolyl, thiadiazolyl, pyrrolidinyl, piperazinyl, piperidinyl, morpholinyl, and tetrahydroyranyl, and wherein alkyl, cycloalkyl and alkoxy are optionally substituted by halogen, and wherein aryl and heterocyclyl moieties are optionally substituted by one or more R²⁴.

Even more preferably each R²³ independently is halogen, cyano, amino, mercapto, C₁-C₄alkyl, C₃-C₆cycloalkyl, C₃-C₆cycloalkyl-C₁-C₄alkylthio, C₁-C₄alkoxy, C₁-C₄alkylthio, phenyl and phenyloxy, and wherein alkyl, cycloalkyl and alkoxy are optionally substituted by halogen, and wherein phenyl is optionally substituted by one or more R²⁴.

Each R²⁴ is independently halogen, cyano, C₁-C₄alkyl, C₁-C₄haloalkyl, C₁-C₄alkoxy or C₁-C₄haloalkoxy, preferably halogen, cyano, methyl, halomethyl, methoxy or halomethoxy, more preferably halogen, methyl or halomethyl.

Preferably R²⁵ is C₁-C₄alkyl or C₁-C₄alkoxy.

The compound of formula I may be a compound of formula I.d

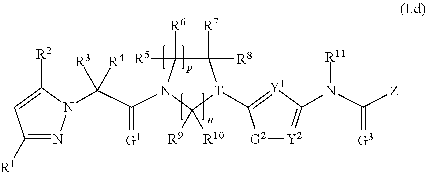

wherein Z is selected from Z1 to Z19

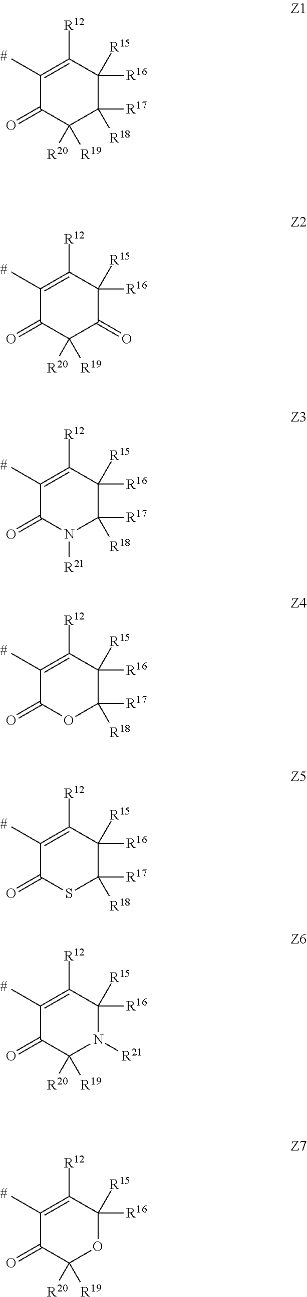

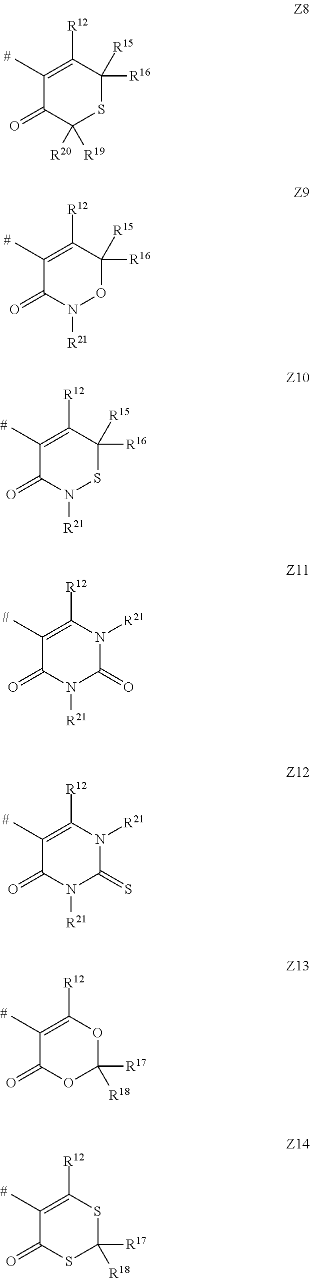

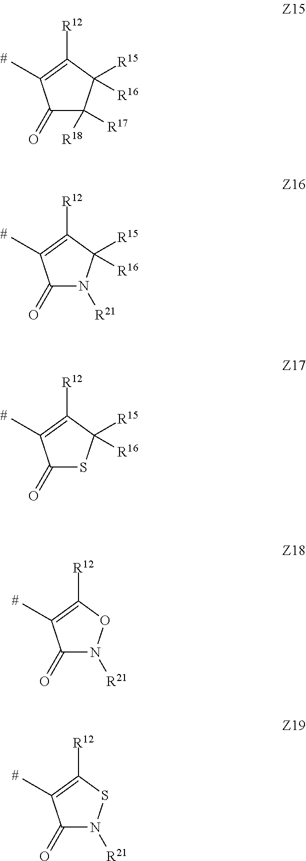

and G¹, G², G³, T, Y¹, Y², n, p, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰ and R²¹ are as defined for the compound of formula I. The preferred substituent definitions are the same as for compounds of formula I. Preferably Z is selected from Z1, Z2, Z3, Z4, Z7, Z8, Z9, Z11, Z12, Z13, Z16, Z17 and Z18.

R¹⁵, R¹⁶, R¹⁷, R¹⁸, R¹⁹ and R²⁰ may form alicyclic and/or heterocyclic rings as described above. Examples of Z in such cases include, but are not limited to, the following

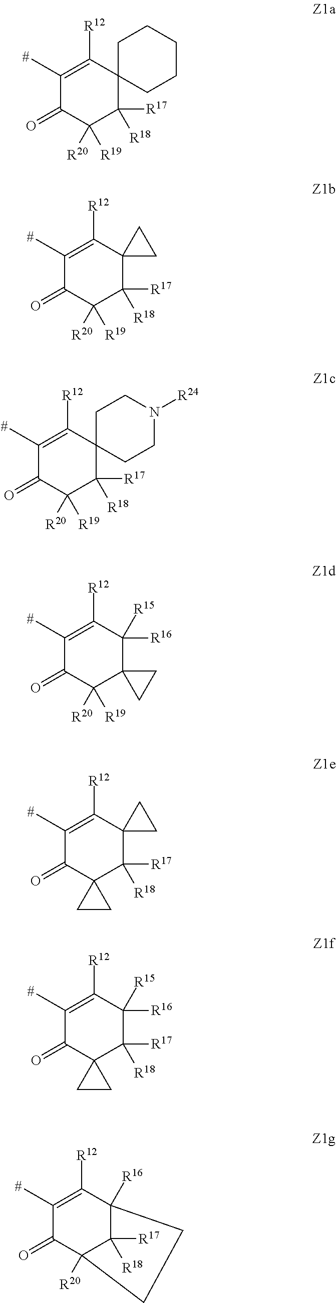

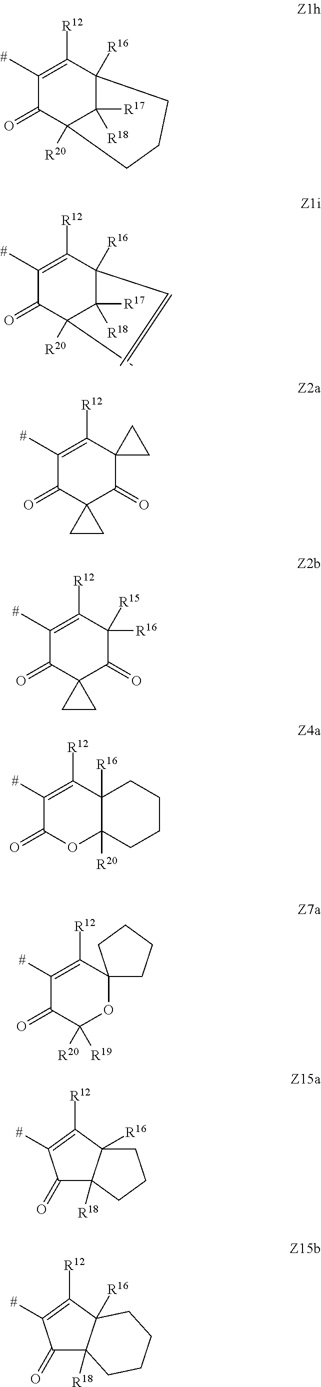

The compound of the invention may be a compound of formula I.d wherein Z is selected from Z1 to Z19, e.g. from Z1, Z2, Z3, Z4, Z7, Z8, Z9, Z11, Z12, Z13, Z16, Z17 and Z18, and wherein Z1 is selected from Z1a-Z1i, Z2 is selected from Z2a and Z2b, Z4 is Z4a, Z7 is Z7a and Z15 is selected from Z15a and Z15b, and wherein R¹⁵, R¹⁶, R¹⁷, R¹⁸, R¹⁹ and R²⁰ each independently are hydrogen, halogen, hydroxyl, amino, cyano, C₁-C₈alkyl, C₂-C₈alkenyl, C₂-C₈alkynyl, C₃-C₈cycloalkyl, C₁-C₈alkoxy, C₁-C₈alkylthio, C₁-C₈alkylsulfonyl, C₁-C₈alkylsulfinyl, aryl, heteroaryl or NHR²², wherein alkyl, alkenyl, alkynyl, cycloalkyl, alkoxy, aryl and heteroaryl are optionally substituted by one or more R²³.

In one group of compounds of the invention G¹, G² and G³ are independently O or S; T is CR¹³ or N; Y¹ is N; Y² is CR¹⁴ or N; A is C(R¹⁵R¹⁶), C(═O), NR²¹, O or S; Q¹ is C(R¹⁷R¹⁸), C(═O), NR²¹, O or S; Q² is C(R¹⁹R²⁰), C(═O), NR²¹, O or S; n is 1 or 2; p is 1; x is 0 or 1; R¹, R², R³, R⁴, R⁵, R⁶, R⁷, R⁸, R⁹, R¹⁰, R¹³ and R¹⁴ each independently are hydrogen, halogen, C₁-C₄alkyl, C₃-C₅cycloalkyl or C₁-C₄haloalkyl; R¹¹ and R²² independently are hydrogen, C₁-C₄alkyl or C₁-C₄alkoxy; R¹² is hydroxyl, O⁻M⁺, OC(═O)R²⁵, amino or NHR²²; M⁺ is a metal cation or ammonium cation, R¹⁵, R¹⁶, R¹⁷, R¹⁸, R¹⁹ and R²⁰ each independently are hydrogen, halogen, hydroxyl, cyano, C₁-C₄alkyl, C₁-C₈alkylcarbonyl, C₃-C₅cycloalkyl, C₁-C₄haloalkyl, C₁-C₄alkoxy, C₁-C₈alkoxycarbonyl, C₁-C₄haloalkoxy, C₁-C₄alkylthio, aryl, arylcarbonyl, heteroaryl or NHR²⁴; and wherein R¹⁵ and R¹⁶, R¹⁷ and R¹⁸ and/or R¹⁹ and R²⁰ together form a saturated three- to six-membered alicyclic or heterocyclic ring; or R¹⁵ and R¹⁷ and/or R¹⁸ and R¹⁹ together form a saturated four- to seven membered alicyclic or heterocyclic ring; and/or R¹⁵ and R¹⁹ together form a saturated four- to seven-membered alicyclic or heterocyclic ring; each R²⁴ independently is hydrogen, C₁-C₄alkyl, C₃-C₅cycloalkyl, C₁-C₄alkylcarbonyl, C₁-C₄haloalkylcarbonyl, C₁-C₄alkylsulfonyl or C₁-C₄ haloalkylsulfonyl; and R²⁵ is C₁-C₄alkyl or C₁-C₄alkoxy.

In another group of compounds of the invention G¹ is O; G² is O or S; G³ is O; T is CR¹³ or N; Y¹ is N; Y² is CR¹⁴ or N; A is C(R¹⁵R¹⁶), C(═O), NR²¹ or O; Q¹ is C(R¹⁷R¹⁸), C(═O), NR²¹ or O; Q² is C(R¹⁹R²⁰), C(═O), NR²¹ or O; n is 1 or 2; p is 1; x is 0 or 1; R¹, R², R³, R⁴, R⁵, R⁶, R⁷, R⁸, R⁹, R¹⁰, R¹³ and R¹⁴ each independently are hydrogen, halogen, C₁-C₄alkyl or C₁-C₄haloalkyl; R¹¹ and R²² are hydrogen or C₁-C₄alkyl; R¹² is hydroxyl, O⁻M⁺, OC(═O)R²⁵ or NHR²²; M⁺ is a metal cation or ammonium cation; R¹⁵, R¹⁶, R¹⁷, R¹⁸, R¹⁹ and R²⁰ each independently are hydrogen, halogen, cyano, C₁-C₄alkyl, C₃-C₅cycloalkyl, C₁-C₄haloalkyl, C₁-C₄alkoxy, C₁-C₈alkoxycarbonyl, C₁-C₄alkylthio, aryl, heteroaryl or NHR²⁴; and wherein R¹⁵ and R¹⁶, R¹⁷ and R¹⁸ and/or R¹⁹ and R²⁰ may together form a saturated three- to six-membered alicyclic or heterocyclic ring; and R²⁴ is hydrogen, C₁-C₄alkyl, C₃-C₅cycloalkyl, C₁-C₄alkylcarbonyl, C₁-C₄haloalkylcarbonyl, C₁-C₄alkylsulfonyl or C₁-C₄ haloalkylsulfonyl.

In another group of compounds of the invention G¹ is O; G² is S; G³ is O; T is CH or N; Y¹ is N; Y² is CH or N; A is C(R¹⁵R¹⁶), C(═O), NH, NCH₃ or O; Q¹ is C(R¹⁷R¹⁸), C(═O), NH, NCH₃ or O; Q² is C(R¹⁹R²⁰), C(═O), NH, NCH₃ or O; n is 1 or 2; p is 1; x is 1; R¹ and R² each independently are hydrogen, methyl, difluoromethyl or trifluoromethyl; R³, R⁴, R⁵, R⁶, R⁷, R⁸, R⁹, R¹⁰ each independently are hydrogen or methyl; R¹¹ is hydrogen or methyl; R¹² is hydroxyl, O⁻M⁺; M⁺ is a metal cation or ammonium cation; R¹⁵, R¹⁶, R¹⁷, R¹⁸, R¹⁹ and R²⁰ each independently are hydrogen, fluoro, cyano, methyl, ethyl, cyclopropyl, cyclobutyl, trifluoromethyl, methoxy, methylthio, phenyl or pyridyl; and wherein R¹⁵ and R¹⁶, R¹⁷ and R¹⁸ and/or R¹⁹ and R²⁰ may together form a cyclopropyl or a cyclobutyl ring.

In another group of compounds of the invention G¹ is O; G² is S; G³ is O; T is CH; Y¹ is N; Y² is CH; A is C(R¹⁵R¹⁶), C(═O) or O; Q¹ is C(R¹⁷R¹⁸), C(═O) or O; Q² is C(R¹⁹R²⁰), C(═O) or O; n is 2; p is 1; x is 1; R¹ and R² each independently are hydrogen, methyl, difluoromethyl or trifluoromethyl; R³, R⁴, R⁵, R⁶, R⁷, R⁸, R⁹, R¹⁰ each independently are hydrogen or methyl; R¹¹ is hydrogen or methyl; R¹² is hydroxyl or O⁻M⁺; M⁺ is a metal cation; R¹⁵, R¹⁶, R¹⁷, R¹⁸, R¹⁹ and R²⁰ each independently are hydrogen, methyl, ethyl, cyclopropyl, trifluoromethyl, methoxy, methylthio or phenyl; and wherein R¹⁵ and R¹⁶, R¹⁷ and R¹⁸ and/or R¹⁹ and R²⁰ may together form a cyclopropyl ring.

In another group of compounds of the invention G¹ is O; G² is S; G³ is O; T is CH; Y¹ is N; Y² is CH; A is C(R¹⁵R¹⁶), or C(═O); Q¹ is C(R¹⁷R¹⁸), or C(═O); Q² is C(R¹⁹R²⁰), or C(═O); n is 2; p is 1; x is 2; R¹ is difluoromethyl or trifluoromethyl; R² is methyl or difluoromethyl; R³, R⁴, R⁵, R⁶, R⁷, R⁸, R⁹, R¹⁰ each independently are hydrogen; R¹¹ is hydrogen; R¹² is hydroxyl; R¹⁵, R¹⁶, R¹⁷, R¹⁸, R¹⁹ and R²⁰ each independently are hydrogen, methyl, cyclopropyl or methylthio; and wherein R¹⁵ and R¹⁶, R¹⁷ and R¹⁸ and/or R¹⁹ and R²⁰ may together form a cyclopropyl ring.

In another group of compounds the compound of the invention is a compound of formula I.d; G¹, G² and G³ are independently O or S; T is CR¹³ or N; Y¹ is N; Y² is CR¹⁴ or N; n is 1 or 2; p is 1; Z is selected from Z1 to Z19 (above); R¹, R², R³, R⁴, R⁵, R⁶, R⁷, R⁸, R⁹, R¹⁰, R¹³ and R¹⁴ each independently are hydrogen, halogen, C₁-C₄alkyl, C₃-C₅cycloalkyl or C₁-C₄haloalkyl; R¹¹ and R²² independently are hydrogen, C₁-C₄alkyl or C₁-C₄alkoxy; R¹² is hydroxyl, O⁻M⁺, OC(═O)R²⁵, amino or NHR²²; M⁺ is a metal cation or ammonium cation, R¹⁵, R¹⁶, R¹⁷, R¹⁸, R¹⁹ and R²⁰ each independently are hydrogen, halogen, hydroxyl, cyano, C₁-C₄alkyl, C₃-C₅cycloalkyl, C₁-C₄haloalkyl, C₁-C₄alkoxy, C₁-C₄haloalkoxy, C₁-C₄alkylthio, aryl, heteroaryl or NHR²⁴; and wherein R¹⁵ and R¹⁶, R¹⁷ and R¹⁸ and/or R¹⁹ and R²⁰ may together form a saturated three- to six-membered alicyclic or heterocyclic ring; or R¹⁵ and R¹⁷ and/or R¹⁸ and R¹⁹ together form a saturated four- to seven membered alicyclic or heterocyclic ring; and/or R¹⁵ and R¹⁹ together form a saturated four- to seven-membered alicyclic or heterocyclic ring; each R²⁴ independently is hydrogen, C₁-C₄alkyl, C₃-C₅cycloalkyl, C₁-C₄alkylcarbonyl, C₁-C₄haloalkylcarbonyl, C₁-C₄alkylsulfonyl or C₁-C₄ haloalkylsulfonyl; and R²⁵ is C₁-C₄alkyl or C₁-C₄alkoxy.

In another group of compounds the compound of the invention is a compound of formula I.d, wherein G¹ is O; G² is S; G³ is O; T is CH; Y¹ is N; Y² is CH; Z is selected from Z1 to Z19 (above); preferably Z1, Z2, Z3, Z4, Z7, Z8, Z9, Z11, Z12, Z13, Z16, Z17 and Z18; n is 2; p is 1; R¹ is difluoromethyl or trifluoromethyl; R² is methyl or difluoromethyl; R³, R⁴, R⁵, R⁶, R⁷, R⁸, R⁹, R¹⁰ each independently are hydrogen; R¹¹ is hydrogen; R¹² is hydroxyl; R¹⁵, R¹⁶, R¹⁷, R¹⁸, R¹⁹ and R²⁰ each independently are hydrogen, methyl, cyclopropyl or methylthio; and wherein R¹⁵ and R¹⁶, R¹⁷ and R¹⁸ and/or R¹⁹ and R²⁰ may together form a cyclopropyl ring.

For the avoidance of doubt, when n is 1 and p is 1 compounds of formula I have the formula according to formula I-A:

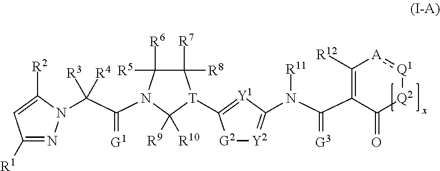

in which R¹, R², R³, R⁴, R⁵, R⁶, R⁷, R⁸, R⁹, R¹⁰, R¹¹, R¹², G¹, G², G³, T, Y¹, Y², A, Q¹, Q² and x have the definitions as described for formula I.

When n is 2 and p is 1, compounds of formula I have the formula according to formula I-B:

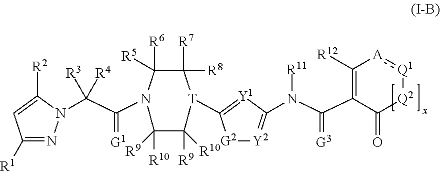

which R¹, R², R³, R⁴, R⁵, R⁶, R⁷, R⁸, R⁹, R¹⁰, R¹¹, R¹², G¹, G², G³, T, Y¹, Y², A, Q¹, Q² and x have the definitions as described for formula I.

When n is 1 and p is 2, compounds of formula I have the formula according to formula I-C:

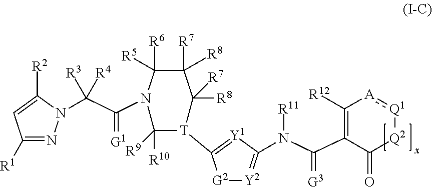

in which R¹, R², R³, R⁴, R⁵, R⁶, R⁷, R⁸, R⁹, R¹⁰, R¹¹, R¹², G¹, G², G³, T, Y¹, Y², A, Q¹, Q² and x have the definitions as described for formula I.

The invention also relates to compounds of formula I-A, formula I-B and formula I-C as shown above.

The invention also relates to compounds of formula I-D:

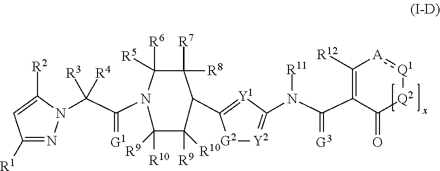

in which R¹, R², R³, R⁴, R⁵, R⁶, R⁷, R⁸, R⁹, R¹⁰, R¹¹, R¹², G², G³, Y¹, Y², A, Q¹, Q² and x have the definitions as described for formula I. Preferred definitions of R¹, R², R³, R⁴, R⁵, R⁶, R⁷, R⁸, R⁹, R¹⁰, R¹¹, R¹², G², G³, Y¹, Y², A, Q¹, Q² and x are as defined for formula I.

The invention also relates to compounds of formula I-E:

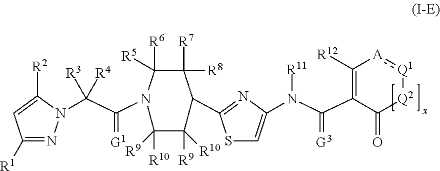

in which R¹, R², R³, R⁴, R⁵, R⁶, R⁷, R⁸, R⁹, R¹⁰, R¹¹, R¹², G³, A, Q¹, Q² and x have the definitions as described for formula I. Preferred definitions of R¹, R², R³, R⁴, R⁵, R⁶, R⁷, R⁸, R⁹, R¹⁰, R¹¹, R¹², G³, A, Q¹, Q² and x are as defined for formula I.

The invention also relates to compounds of formula I-F:

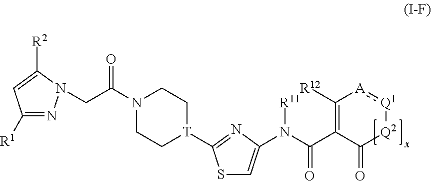

wherein T is CH or N, preferably CH; R¹¹ is CH₃ or H; and R¹, R², R¹², A, Q¹, Q² and x have the definitions as described for formula I. Preferred definitions of R¹, R², R¹², A, Q¹, Q² and x are as defined for formula I.

The invention also relates to compounds of formula I-G:

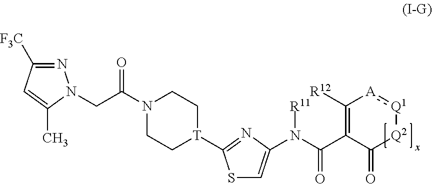

wherein T is CH or N, preferably CH; R¹¹ is CH₃ or H; and R¹², A, Q¹, Q² and x have the definition as described for formula I. Preferred definitions of R¹², A, Q¹, Q² and x are as defined for formula I.

The invention also relates to compounds of formula I-H:

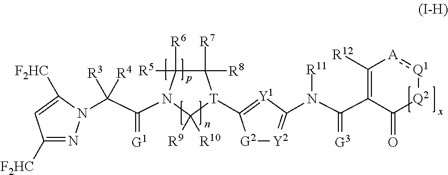

in which R³, R⁴, R⁵, R⁶, R⁷, R⁸, R⁹, R¹⁰, R¹¹, R¹², G¹, G², G³, Y¹, Y², A, Q¹, Q², n, p and x have the definitions as described for formula I. Preferred definitions of R¹, R², R³, R⁴, R⁵, R⁶, R⁷, R⁸, R⁹, R¹⁰, R¹¹, R¹², G¹, G², G³, Y¹, Y², A, Q¹, Q², n, p and x are as defined for formula I.

The invention also relates to compounds of formula I-I:

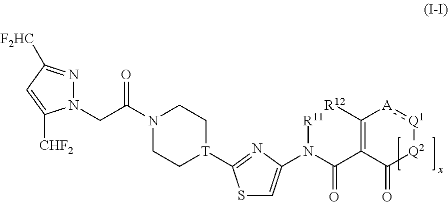

wherein T is CH or N, preferably CH; R¹¹ is CH₃ or H; and R¹², A, Q¹, Q² and x have the definition as described for formula I. Preferred definitions of R¹², A, Q¹, Q² and x are as defined for formula I.

The invention includes compounds of formula II.b:

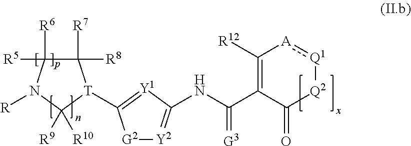

wherein R is hydrogen, a protecting group, such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl, and G², G³, T, Y¹, Y², n, p, R⁵, R⁶, R⁷, R⁸, R⁹, R¹⁰, R¹², A, Q¹, Q² and x are as defined for a compound of formula I. These compounds, including salts and N-oxides thereof, are useful as intermediates in the synthesis of compounds of formula I. Preferred definitions of G², G³, T, Y¹, Y², n, p, R⁵, R⁶, R⁷, R⁸, R⁹, R¹⁰, R¹², A, Q¹, Q² and x are as defined for formula I.

The invention also includes compounds of formula III

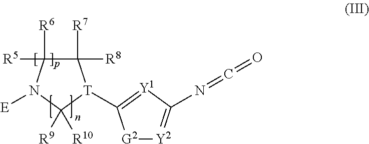

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

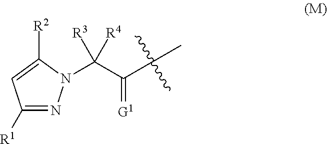

and G¹, G², T, Y¹, Y², n, p, R¹, R², R³, R⁴, R⁵, R⁶, R⁷, R⁸, R⁹ and R¹⁰ are as defined for a compound of formula I. These compounds, including salts and N-oxides thereof, are useful as intermediates in the synthesis of compounds of formula I. Preferred definitions of G¹, G², T, Y¹, Y², n, p, R¹, R², R³, R⁴, R⁵, R⁶, R⁷, R⁸, R⁹ and R¹⁰ are as defined for formula I.

The invention also includes compounds of formula IV

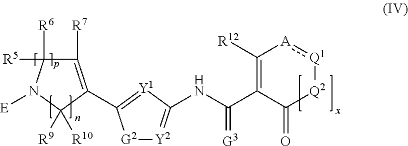

wherein E is hydrogen, a protecting group such as alkylcarbonyl, benzyl or alkoxycarbonyl, e.g. C₁-C₄ alkylcarbonyl, benzyl or C₁-C₄ alkoxycarbonyl, in particular acetyl, benzyl or tert-butoxycarbonyl; or group M

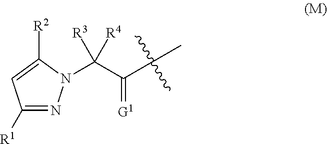

and G¹, G², G³, Y¹, Y², n, p, R¹, R², R³, R⁴, R⁵, R⁶, R⁷, R⁹, R¹⁰, R¹², A, Q¹, Q² and x are as defined for a compound of formula I. These compounds, including salts and N-oxides thereof, are useful as intermediates in the synthesis of compounds of formula I. Preferred definitions of G¹, G², G³, Y¹, Y², n, p, R¹, R², R³, R⁴, R⁵, R⁶, R⁷, R⁹, R¹⁰, R¹², A, Q¹, Q² and x are as defined for formula I.

The invention also includes compounds of formula V

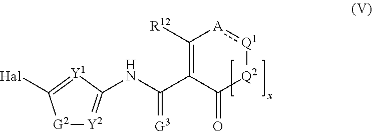

wherein Hal is halogen and G², G³, Y¹, Y², R¹², A, Q¹, Q² and x are as defined for a compound of formula I. These compounds, including salts and N-oxides thereof, are useful as intermediates in the synthesis of compounds of formula I. Preferred definitions of G², G³, Y¹, Y², R¹², A, Q¹, Q² and x are as defined for formula I.

Preferred individual compounds of formula I are:
2,6-Dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
4,4-Dimethyl-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
3,3,5,5-Tetramethyl-2,4,6-trioxo-cyclohexanecarboxylic acid (2-{1-[2-(5-methyl-3-trifluoromethyl-pyrazol-1-yl)-acetyl]-piperidin-4-yl}-thiazol-4-yl)-amide;
5,7-Dioxo-spiro[2.5]octane-6-carboxylic acid (2-{1-[2-(5-methyl-3-trifluoromethyl-pyrazol-1-yl)-acetyl]-piperidin-4-yl}-thiazol-4-yl)-amide;
3-Methyl-3-methylsulfanyl-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
3,3,5-Trimethyl-5-methylsulfanyl-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
4-(1-Methylsulfanyl-cyclopropyl)-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
3,3-Dimethyl-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
4-(2-Methyl-1-methylsulfanyl-cyclopropyl)-2,6-dioxo-cyclohexanecarboxylic acid (2-{1-[2-(5-methyl-3-trifluoromethyl-pyrazol-1-yl)-acetyl]-piperidin-4-yl}-thiazol-4-yl)-amide;
2,4-Dioxo-bicyclo[3.2.1]octane-3-carboxylic acid (2-{1-[2-(5-methyl-3-trifluoromethyl-pyrazol-1-yl)-acetyl]-piperidin-4-yl}-thiazol-4-yl)-amide; and
4,6-Dioxo-spiro[2.5]octane-5-carboxylic acid (2-{1-[2-(5-methyl-3-trifluoromethyl-pyrazol-1-yl)acetyl]-piperidin-4-yl}-thiazol-4-yl)-amide.

Compounds of the present invention can be made as shown in the following schemes. Throughout this description, the group M, wherein R¹, R², R³, R⁴ and G¹ are as defined for formula I, stands for:

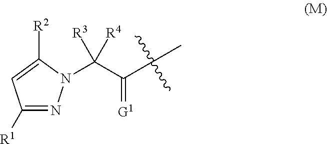

The compounds of formula II.a, wherein R⁵, R⁶, R⁷, R⁸, R⁹, R¹⁰, A, G², G³, Q¹, Q², T, Y¹, Y², n, p and x are as defined for formula I and E is hydrogen, a protecting group such as acetyl, benzyl or tert-butoxycarbonyl or a group M, can be obtained by transformation of a compound of formula III, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a compound of formula VI, wherein A, Q¹, Q² and x are as defined for formula I and a base, such as sodium hydride, potassium hydride, pyridine, 4-dimethylaminopyridine or triethylamine.

This is shown in Scheme 1.

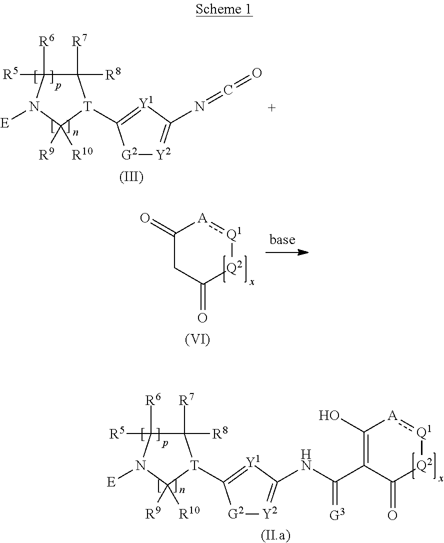

The compounds of formula III, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, can be obtained by transformation of a compound of formula VII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with 1,1′-carbonyldiimidazole (CDI). This is shown in Scheme 2.

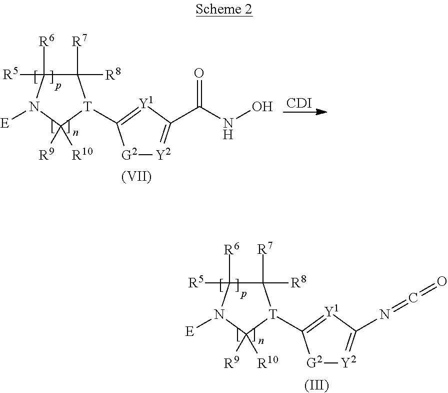

The compounds of formula VII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, can be obtained by transformation of a compound of formula VIII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with hydroxylamine or hydroxylamine hydrochloride and an activating reagent such as 1,1′-carbonyldiimidazole (CDI). This is shown in Scheme 3.

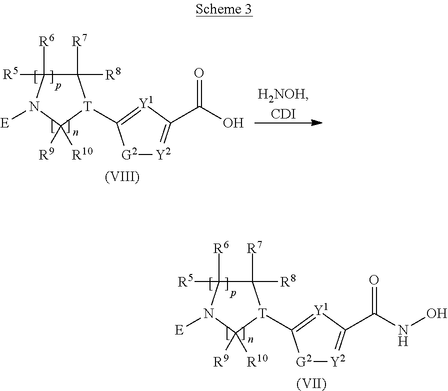

Alternatively, the compounds of formula VII, wherein R⁵, R⁶, R⁷, R⁸, R⁹, R¹⁰, G², T, Y¹, Y² n and p are as defined for formula I and E is hydrogen, a protecting group such as acetyl, benzyl or tert-butoxycarbonyl or a group M, can be obtained by transformation of a compound of formula IX, wherein R⁵, R⁶, R⁷, R⁸, R⁹, R¹⁰, G², T, Y¹, Y², n and p are as defined for formula I, Hal is halogen, preferably chloro or bromo, and E is hydrogen, a protecting group such as acetyl, benzyl or tert-butoxycarbonyl or a group M, with hydroxylamine or hydroxylamine hydrochloride. This is shown in Scheme 4.

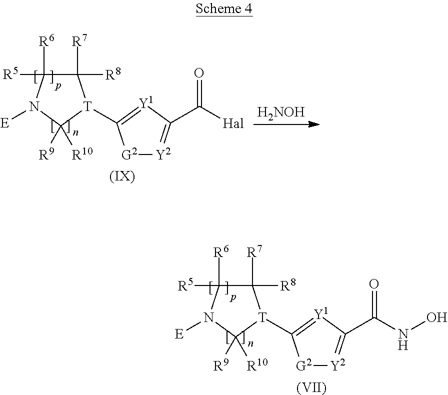

The compounds of formula VIII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, can be obtained by transformation of a compound of formula X, wherein R⁵, R⁶, R⁷, R⁸, R⁹, R¹⁰, G², T, Y¹, Y², n and p are as defined for formula I, R²⁶ is C₁-C₄alkyl or optionally substituted aryl and E is hydrogen, a protecting group such as acetyl, benzyl or tert-butoxycarbonyl or a group M, with a base, such as sodium hydroxide, potassium hydroxide or lithium hydroxide. This is shown in Scheme 5.

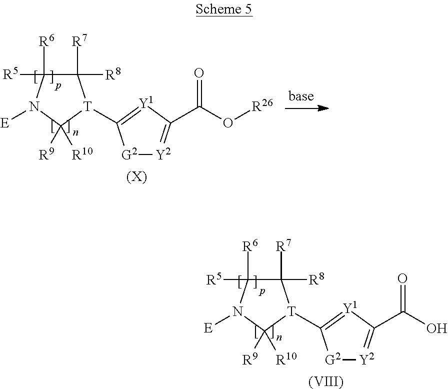

The compounds of formula IX, wherein R⁵, R⁶, R⁷, R⁸, R⁹, R¹⁰, G², T, Y¹, Y², n and p are as defined for formula I, Hal is halogen, preferably chloro or bromo, and E is hydrogen, a protecting group such as acetyl, benzyl or tert-butoxycarbonyl or a group M, can be obtained by transformation of a compound of formula VIII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a halogenation reagent, such as thionyl chloride or thionyl bromide. This is shown in Scheme 6.

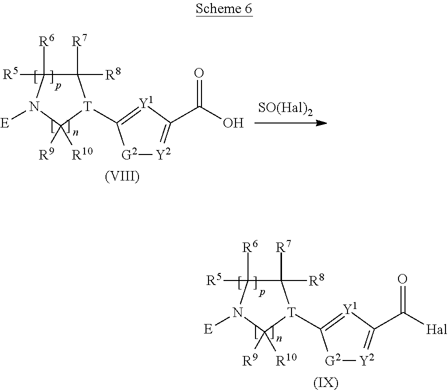

Alternatively, the compounds of formula II.a, wherein R⁵, R⁶, R⁷, R⁸, R⁹, R¹⁰, A, G², G³, Q¹, Q², T, Y¹, Y², n, p and x are as defined for formula I and E is hydrogen, a protecting group such as acetyl, benzyl or tert-butoxycarbonyl or a group M, can be obtained by transformation of a compound of formula XI, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, with a compound of formula XII, wherein A, Q¹, Q² and x are as defined for formula I and R²⁷ is hydroxy or halogen, preferably chloro or bromo, and a base, such as pyridine, 4-dimethylaminopyridine or triethylamine and, provided R²⁰ is hydroxy, an additional activating reagent, such as BOP or CDI. This is shown in Scheme 7.

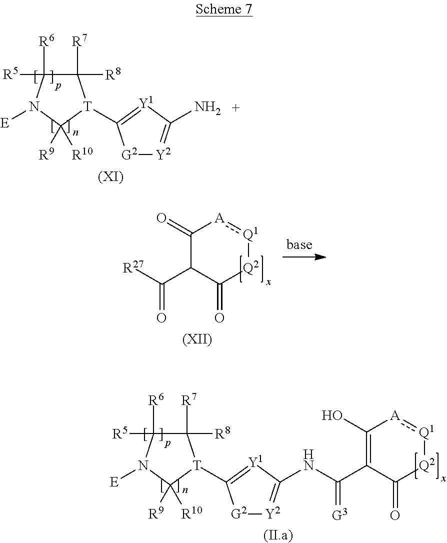

The compounds of formula XI, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M, can be obtained by transformation of a compound of formula VIII, wherein R⁵, R⁶, R⁷, R⁸, R⁹, R¹⁰, G², T, Y¹, Y², n and p are as defined for formula I and E is hydrogen, a protecting group such as acetyl, benzyl or tert-butoxycarbonyl or a group M with an azide, such as diphenyl phosphoryl azide and subsequent Curtius rearrangement of the resulting acyl azide with an alcohol R²⁶—OH, wherein R²⁶ is C₁-C₄alkyl or optionally substituted aryl, and following carbamate cleavage with a mineral acid, such as hydrochloric acid, sulfuric acid or an organic acid, such as trifluoroacetic acid. This is shown in Scheme 8.

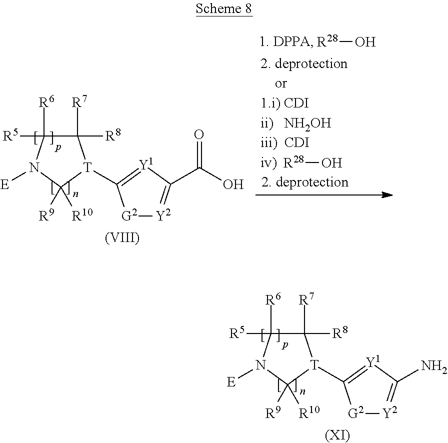

The compounds of formula XIII, wherein R⁵, R⁶, R⁷, R⁹, R¹⁰, A, G², G³, Q¹, Q², Y¹, Y², n, p and x are as defined for formula I and E is hydrogen, a protecting group such as acetyl, benzyl or tert-butoxycarbonyl or a group M, can be obtained by transformation of a compound of formula IV.a, wherein R⁵, R⁶, R⁷, R⁹, R¹⁰, A, G², G³, Q¹, Q², Y¹, Y², n, p and x are as defined for formula I and E is hydrogen, a protecting group such as acetyl, benzyl or tert-butoxycarbonyl or a group M, with hydrogen and a catalyst, such as palladium on charcoal or raney-nickel. This is shown in Scheme 9.

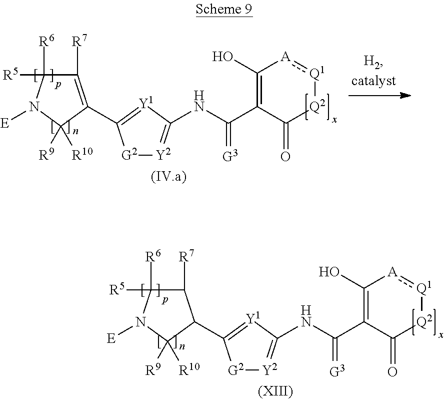

The compounds of formula IV.a, wherein R⁵, R⁶, R⁷, R⁹, R¹⁰, A, G², G³, Q¹, Q², Y¹, Y², n, p and x are as defined for formula I and E is hydrogen, a protecting group such as acetyl, benzyl or tert-butoxycarbonyl or a group M, can be obtained by transformation of a compound of formula XIV, wherein R⁵, R⁶, R⁷, R⁹, R¹⁰, n and p are as defined for formula I, R²⁸ is B(OH)₂ or an ester of such a boronic acid and E is hydrogen, a protecting group such as acetyl, benzyl or tert-butoxycarbonyl or a group M, with a compound of formula V.a, wherein A, G², G³, A, Q¹, Q², Y¹, Y² and x are as defined for formula I and Hal is halogen, preferably chloro, bromo or iodo, and a transition metal, such as tetrakis(triphenylphosphine)palladium, and a ligand. This is shown in Scheme 10.

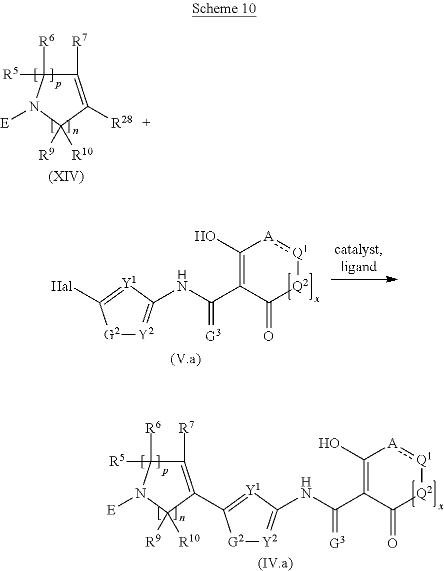

The compounds of formula V.a, wherein A, G², G³, Q¹, Q², Y¹, Y² and x are as defined for formula I and Hal is halogen, preferably chloro, bromo or iodo, can be obtained by transformation of a compound of formula XV, wherein G², Y¹ and Y² are as defined for formula I and Hal is halogen, preferably chloro, bromo or iodo, with a compound of formula VI, wherein A, Q¹, Q² and x are as defined for formula I, and a base, such as sodium hydride, potassium hydride, pyridine, 4-dimethylaminopyridine or triethylamine. This is shown in Scheme 11.

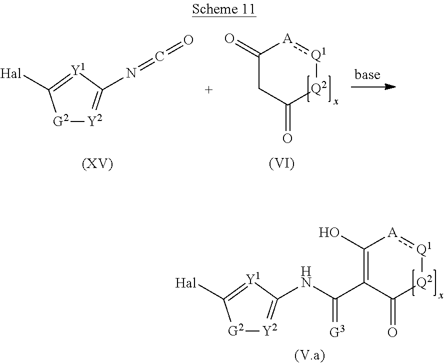

The compounds of formula XV, wherein G², Y¹ and Y² are as defined for formula I and Hal is halogen, preferably chloro, bromo or iodo, can be obtained by transformation of a compound of formula XVI, wherein G², Y¹ and Y² are as defined for formula I and Hal is halogen, preferably chloro, bromo or iodo, with 1,1′carbonyldiimidazole. This is shown in Scheme 12.

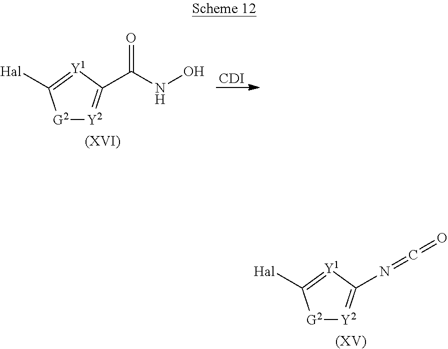

The compounds of formula XVI, wherein G², Y¹ and Y² are as defined for formula I and Hal is halogen, preferably chloro, bromo or iodo, can be obtained by transformation of a compound of formula XVII, wherein G², Y¹ and Y² are as defined for formula I and Hal is halogen, preferably chloro, bromo or iodo, with hydroxylamine or hydroxylamine hydrochloride and an activating reagent such as 1,1′-carbonyldiimidazole (CDI). This is shown in Scheme 13.

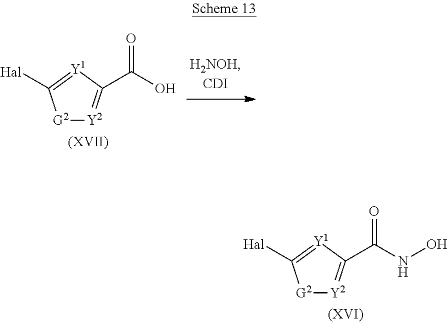

Alternatively the compounds of formula XVI, wherein G², Y¹ and Y² are as defined for formula I and Hal is halogen, preferably chloro, bromo or iodo, can be obtained by transformation of a compound of formula XVII, wherein G², Y¹ and Y² are as defined for formula I and Hal is halogen, preferably chloro, bromo or iodo, with hydroxylamine or hydroxylamine hydrochloride. This is shown in Scheme 14.

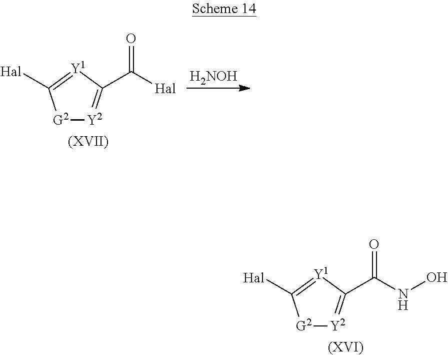

Surprisingly, it has now been found that the novel compounds of formula I have, for practical purposes, a very advantageous level of biological activity for protecting plants against diseases that are caused by fungi.

The compounds of formula I can be used in the agricultural sector and related fields of use e.g. as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic micro-organisms.

It is also possible to use compounds of formula I as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula I before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds according to present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula I are, for example, effective against the phytopathogenic fungi of the following classes: Fungi imperfecti (e.g. Alternaria spp.), Basidiomycetes (e.g. Corticium spp., Ceratobasidium spp., Waitea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp). Outstanding activity is observed against downy mildew (e.g. Plasmopara viticola) and late blight (e.g. Phytophthora infestans). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

Within the scope of present invention, target crops and/or useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as turf and ornamentals.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a plant as used herein is intended to embrace the place on which the plants are growing, where the plant propagation materials of the plants are sown or where the plant propagation materials of the plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula I may be used in the form of (fungicidal) compositions for controlling or protecting against phytopathogenic microorganisms, comprising as active ingredient at least one compound of formula I or of at least one preferred individual compound as above-defined, in free form or in agrochemically usable salt form, and at least one of the above-mentioned adjuvants.

The invention provides a fungicidal composition comprising at least one compound formula I an agriculturally acceptable carrier and optionally an adjuvant. An agricultural acceptable carrier is for example a carrier that is suitable for agricultural use. Agricultural carriers are well known in the art. Preferably said fungicidal compositions may comprise at least one additional fungicidal active ingredient in addition to the compound of formula I.

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities. Examples of suitable additional active ingredients include the following: Azoxystrobin (131860-33-8), Dimoxystrobin (149961-52-4), Enestrobin (238410-11-2), Fluoxastrobin (193740-76-0), Kresoxim-methyl (143390-89-0), Metominostrobin (133408-50-1), Orysastrobin (248593-16-0), Picoxystrobin (117428-22-5), Pyraclostrobin (175013-18-0), trifloxystrobin (141517-21-7), Azaconazole (60207-31-0), Bromuconazole (116255-48-2), Cyproconazole (94361-06-5), Difenoconazole (119446-68-3), Diniconazole (83657-24-3), Diniconazole-M (83657-18-5), Epoxiconazole (13385-98-8), Fenbuconazole (114369-43-6), Fluquinconazole (136426-54-5), Flusilazole (85509-19-9), Flutriafol (76674-21-0), Hexaconazole (79983-71-4), Imazalil (58594-72-2), Imibenconazole (86598-92-7), Ipconazole (125225-28-7), Metconazole (125116-23-6), Myclobutanil (88671-89-0), Oxpoconazole (174212-12-5), Pefurazoate (58011-68-0), Penconazole (66246-88-6), Prochloraz (67747-09-5), Propiconazole (60207-90-1), Prothioconazole (178928-70-6), Simeconazole (149508-90-7), Tebuconazole (107534-96-3), Tetraconazole (112281-77-3), Triadimefon (43121-43-3), Triadimenol (55219-65-3), Triflumizole (99387-89-0), Triticonazole (131983-72-7), Diclobutrazol (76738-62-0), Etaconazole (60207-93-4), Fluconazole (86386-73-4), Fluconazole-cis (112839-32-4), Thiabendazole (148-79-8), Quinconazole (103970-75-8), Fenpiclonil (74738-17-3), Fludioxonil (131341-86-1), Cyprodinil (121552-61-2), Mepanipyrim (110235-47-7), Pyrimethanil (53112-28-0), Aldimorph (91315-15-0), Dodemorph (1593-77-7), Fenpropimorph (67564-91-4), Tridemorph (81412-43-3), Fenpropidin (67306-00-7), Spiroxamine (118134-30-8), Isopyrazam (881685-58-1), Sedaxane (874967-67-6), Bixafen (581809-46-3), Penthiopyrad (183675-82-3), Fluxapyroxad (907204-31-3), Boscalid (188425-85-6), Penflufen (494793-67-8), Fluopyram (658066-35-4), Mandipropamid (374726-62-2), Benthiavalicarb (413615-35-7), Dimethomorph (110488-70-5), Chlorothalonil (1897-45-6), Fluazinam (79622-59-6), Dithianon (3347-22-6), Metrafenone (220899-03-6), Tricyclazole (41814-78-2), Mefenoxam (70630-17-0), Metalaxyl (57837-19-1), Acibenzolar (126448-41-7) (Acibenzolar-5-methyl (126448-41-7)), Mancozeb (8018-01-7), Ametoctradine (865318-97-4) Cyflufenamid (180409-60-3), and Kresoxim-methyl (143390-89-0), Ipconazole (125225-28-7), Amisulbrom (348635-87-0), Cyflufenamid (180409-60-3), Ethaboxam (16650-77-3), Fluopicolide (239110-15-7), Fluthianil (304900-25-2), Isotianil (224049-04-1), Proquinazid (189278-12-4), Valiphenal (283159-90-0), 1-methyl-cyclopropene (3100-04-7), Trifloxystrobin (141517-21-7), Sulfur (7704-34-9), Copper ammoniumcarbonate (CAS 33113-08-5); Copper oleate (CAS1120-44-1); Folpet (133-07-3), Quinoxyfen (124495-18-7), Captan (133-06-2), Fenhexamid (126833-17-8), Glufosinate and its salts (51276-47-2, 35597-44-5 (S-isomer)), Glyphosate (1071-83-6) and its salts (69254-40-6 (Diammonium), 34494-04-7 (Dimethylammonium), 38641-94-0 (Isopropylammonium), 40465-66-5 (Monoammonium), 70901-20-1 (Potassium), 70393-85-0 (Sesquisodium), 81591-81-3 (Trimesium)), 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]-amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolidin-3-yl]-pyridine.

Another aspect of invention is related to the use of a compound of formula I or of a preferred individual compound as above-defined, of a composition comprising at least one compound of formula I or at least one preferred individual compound as above-defined, or of a fungicidal mixture comprising at least one compound of formula I or at least one preferred individual compound as above-defined, in admixture with other fungicides, as described above, for controlling or preventing infestation of plants, e.g. useful plants such as crop plants, propagation material thereof, e.g. seeds, harvested crops, e.g. harvested food crops, or non-living materials by phytopathogenic microorganisms, preferably fungal organisms.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a compound of formula I or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula I, or an agrochemical composition which contains at least one of said compounds, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula I, and, if desired, a solid or liquid adjuvant or monomers for encapsulating the compound of formula I, may be prepared in a known manner, typically by intimately mixing and/or grinding the compound with extenders, for example solvents, solid carriers and, optionally, surface active compounds (surfactants).

The agrochemical formulations and/or compositions will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Advantageous rates of application are normally from 5 g to 2 kg of active ingredient (a.i.) per hectare (ha), preferably from 10 g to 1 kg a.i./ha, most preferably from 20 g to 600 g a.i./ha.

When used as seed drenching agent, convenient dosages are from 10 mg to 1 g of active substance per kg of seeds.

Whereas it is preferred to formulate commercial products as concentrates, the end user will normally use dilute formulations.

The following non-limiting example illustrates the above-described invention in more detail.

### Example 1

### This Example Illustrates the Preparation of 2-hydroxy-6-oxo-cyclohex-1-enecarboxylic acid (2-{1-[2-(5-methyl-3-trifluoromethyl-pyrazol-1-yl)-acetyl]-piperidin-4-yl}-thiazol-4-yl)-amide

### a) Preparation of 2-{1-[2-(5-methyl-3-trifluoromethyl-pyrazol-1-yl)-acetyl]-piperidin-4-yl}-thiazole-4-carboxylic acid ethyl ester

To a solution of (5-methyl-3-trifluoromethyl-pyrazol-1-yl)-acetic acid (9.1 g, 36.1 mmol) in DMF (100 mL) is added diisopropylethylamine (45 mL, 216 mmol), followed by O-(benzotriazol-1-yl)-N,N,N′,N′-tetramethyluronium tetrafluoroborate (15.5 g, 39.7 mmol). After stirring 15 min at RT, 2-piperidin-4-yl-thiazole-4-carboxylic acid ethyl ester hydrochloride (10 g, 36.1 mmol) is added to the reaction mixture. After stirring overnight at RT, solvent is evaporated and the resulting yellow oil is dissolved in ethylacetate (300 mL), washed with saturated aqueous sodium bicarbonate solution (300 mL), 1M HCl solution (300 mL), and brine (100 mL). The organic layer is dried over sodium sulfate, filtered, and evaporated under reduced pressure. The crude mixture is purified by column chromatography on silica gel (dichloromethane/methanol 10:1) to give 2-{1-[2-(5-methyl-3-trifluoromethyl-pyrazol-1-yl)-acetyl]-piperidin-4-yl}-thiazole-4-carboxylic acid ethyl ester (13.6 g, 88%). ¹H-NMR (400 MHz, CDCl₃): δ=1.40 (t, 3H), 1.70-1.85 (m, 2H), 2.16-2.30 (m, 2H), 2.32 (s, 3H), 2.79-2.89 (m, 1H), 3.22-3.43 (m, 1H), 4.03-4.12 (m, 1H), 4.42 (q, 3H), 4.54-4.69 (m, 1H), 4.93-5.08 (2d, 2H (diastereotopic)), 6.35 (s, 1H), 8.10 (br, 1H). MS: m/z=209 (M+1).

### b) Preparation of 2-{1-[2-(5-methyl-3-trifluoromethyl-pyrazol-1-yl)-acetyl]-piperidin-4-yl}-thiazole-4-carboxylic acid

To a solution of 2-{1-[2-(5-methyl-3-trifluoromethyl-pyrazol-1-yl)-acetyl]-piperidin-4-yl}-thiazole-4-carboxylic acid ethyl ester (2.67 g, 6.2 mmol) in THF (20 mL) is added aqueous solution of sodium hydroxide (2 M, 4.65 mL, 9.3 mmol) at RT. After stirring 3 h at RT, the reaction mixture is acidified with 2M aqueous solution of HCl until pH 2-3, and the solution is extracted with ethylacetate (20 mL). The aqueous layer is re-extracted with ethylacetate (20 mL) and the combined organic layers are washed with brine (10 mL), dried over sodium sulfate, filtered, and evaporated under reduced pressure to give 2-{1-[2-(5-methyl-3-trifluoromethyl-pyrazol-1-yl)-acetyl]-piperidin-4-yl}-thiazole-4-carboxylic acid (2.33 g, 94%), which can be used in the next step without further purification. ¹H-NMR (400 MHz, d₆-acetone): δ=1.69-1.82 (m, 1H), 1.87-2.02 (m, 1H), 2.16-2.37 (m, 2H), 2.38 (s, 3H), 2.89-2.99 (m, 1H), 3.38-3.48 (m, 2H), 4.14-4.22 (m, 1H), 4.50-4.69 (m, 1H), 5.20-5.36 (2d, 2H (diastereotopic)), 6.41 (s, 1H), 8.34 (s, 1H). MS: m/z=403 (M+1).

### c) Preparation of 2-{1-[2-(5-methyl-3-trifluoromethyl-pyrazol-1-yl)-acetyl]-piperidin-4-yl}-thiazole-4-carboxylic acid hydroxyamide

To a suspension of 2-{1-[2-(5-methyl-3-trifluoromethyl-pyrazol-1-yl)-acetyl]-piperidin-4-yl}-thiazole-4-carboxylic acid (1.61 g, 4 mmol) in CH₃CN (15 mL) was added portionwise 1,1-carbonyldiimidazole (0.712 g, 4.4 mmol) at RT. After stirring for 2 h at RT, hydroxylamine hydrochloride (0.556 g, 8 mmol) was added to the solution. The reaction mixture was heated to reflux, stirred overnight at reflux and then concentrated. The solid residue was dissolved in aqueous ammonium chloride solution (50 mL) and ethyl acetate (50 mL) and stirred for 10 min. The aqueous phase was extracted once more with ethyl acetate (50 mL). The combined organic phases were washed with 1M HCl and brine, dried over magnesium sulfate, and evaporated under reduced pressure to give 2-{1-[2-(5-methyl-3-trifluoromethyl-pyrazol-1-yl)-acetyl]-piperidin-4-yl}-thiazole-4-carboxylic acid hydroxyamide (1.37 g, 80%). ¹H-NMR (400 MHz, d₄-methanol): δ=1.76-1.87 (m, 1H), 1.91-2.02 (m, 1H), 2.19-2.30 (m, 2H), 2.33 (s, 3H), 2.95-3.03 (m, 1H), 3.39-3.47 (m, 2H), 4.09-4.14 (m, 1H), 4.50-4.58 (m, 1H), 5.19-5.30 (2d, 2H (diastereotopic)), 6.44 (s, 1H), 8.12 (s, 1H). MS: m/z=418 (M+1).

### d) Preparation of 2-hydroxy-6-oxo-cyclohex-1-enecarboxylic acid (2-{1-[2-(5-methyl-3-trifluoromethyl-pyrazol-1-yl)-acetyl]-piperidin-4-yl}-thiazol-4-yl)-amide

To a solution of 2-{1-[2-(5-methyl-3-trifluoromethyl-pyrazol-1-yl)-acetyl]-piperidin-4-yl}-thiazole-4-carboxylic acid hydroxyamide (0.208 g, 0.5 mmol) in dichloroethane (2 mL) was added 1,1-carbonyldiimidazole (97 mg, 0.6 mmol) at RT. The reaction mixture was heated to 80° C. for 2 h, cooled to RT and a solution of cyclohexane-1,3-dione (56 mg, 0.5 mmol) and Et₃N (50.6 mg, 0.5 mmol) in dichloroethane (0.5 mL) was added by syringe followed by 4 drops of 1-butyl-3-methylimidazolium methylsulfate. After stirring at 80° C. for 2 h, the reaction mixture was cooled to RT, diluted with dichloromethane, washed with 1M HCl and water, dried over MgSO₄ and concentrated to give 248 mg of a yellow oil. The crude mixture was purified by column chromatography on silica gel (toluene, ethanol, dioxane, triethylamine, water 100:40:20:20:5) to give 2-hydroxy-6-oxo-cyclohex-1-enecarboxylic acid (2-{1-[2-(5-methyl-3-trifluoromethyl-pyrazol-1-yl)-acetyl]-piperidin-4-yl}-thiazol-4-yl)-amide (136 mg, 53%). m.p. 102-105° C. ¹H-NMR (400 MHz, CDCl₃): δ=1.69-1.83 (m, 2H), 1.98-2.08 (m, 2H), 2.10-2.23 (m, 2H), 2.32 (s, 3H), 2.52-2.59 (t, 2H), 2.67-2.72 (t, 2H), 2.89-2.99 (m, 1H), 3.15-3.24 (m, 1H), 3.25-3.35 (m, 1H), 3.97-4.06 (m, 1H), 4.48-4.57 (m, 1H), 4.99 (s, 2H), 6.33 (s, 1H), 7.43 (s, 1H), 12.20 (s, 1H), 17.35 (s, 1H). MS: m/z=512 (M+1).

Table 1 below illustrates examples of individual compounds of formula I according to the invention.

TABLE 1
individual compounds of formula I according to the invention

| Comp. | | | | | | | | | |
|---|---|---|---|---|---|---|---|---|---|
| No. | R¹ | R² | G¹ | T | Y¹ | G² | Y² | G³ | R¹¹ |
| 001 | F₃C | H₃C | O | CH | N | S | CH | O | H |
| 002 | F₃C | H₃C | O | CH | N | S | CH | O | CH₃ |
| 003 | F₃C | H₃C | O | CH | N | S | CH | S | H |
| 004 | F₃C | H₃C | O | CH | N | S | CH | S | CH₃ |
| 005 | F₃C | H₃C | O | CH | N | S | N | O | H |
| 006 | F₃C | H₃C | O | CH | N | S | N | O | CH₃ |
| 007 | F₃C | H₃C | O | CH | N | S | N | S | H |
| 008 | F₃C | H₃C | O | CH | N | S | N | S | CH₃ |
| 009 | F₃C | H₃C | O | CH | N | O | CH | O | H |
| 010 | F₃C | H₃C | O | CH | N | O | CH | O | CH₃ |
| 011 | F₃C | H₃C | O | CH | N | O | CH | S | H |
| 012 | F₃C | H₃C | O | CH | N | O | CH | S | CH₃ |
| 013 | F₃C | H₃C | O | N | N | S | CH | O | H |
| 014 | F₃C | H₃C | O | N | N | S | CH | O | CH₃ |
| 015 | F₃C | H₃C | O | N | N | S | CH | S | H |
| 016 | F₃C | H₃C | O | N | N | S | CH | S | CH₃ |
| 017 | F₃C | H₃C | O | N | N | S | N | O | H |
| 018 | F₃C | H₃C | O | N | N | S | N | O | CH₃ |
| 019 | F₃C | H₃C | O | N | N | S | N | S | H |
| 020 | F₃C | H₃C | O | N | N | S | N | S | CH₃ |
| 021 | F₃C | H₃C | O | N | N | O | CH | O | H |
| 022 | F₃C | H₃C | O | N | N | O | CH | O | CH₃ |
| 023 | F₃C | H₃C | O | N | N | O | CH | S | H |
| 024 | F₃C | H₃C | O | N | N | O | CH | S | CH₃ |
| 025 | F₃C | H₃C | S | CH | N | S | CH | O | H |
| 026 | F₃C | H₃C | S | CH | N | S | CH | O | CH₃ |
| 027 | F₃C | H₃C | S | CH | N | S | CH | S | H |
| 028 | F₃C | H₃C | S | CH | N | S | CH | S | CH₃ |
| 029 | F₃C | H₃C | S | CH | N | S | N | O | H |
| 030 | F₃C | H₃C | S | CH | N | S | N | O | CH₃ |
| 031 | F₃C | H₃C | S | CH | N | S | N | S | H |
| 032 | F₃C | H₃C | S | CH | N | S | N | S | CH₃ |
| 033 | F₃C | H₃C | S | CH | N | O | CH | O | H |
| 034 | F₃C | H₃C | S | CH | N | O | CH | O | CH₃ |
| 035 | F₃C | H₃C | S | CH | N | O | CH | S | H |
| 036 | F₃C | H₃C | S | CH | N | O | CH | S | CH₃ |
| 037 | F₃C | H₃C | S | N | N | S | CH | O | H |
| 038 | F₃C | H₃C | S | N | N | S | CH | O | CH₃ |
| 039 | F₃C | H₃C | S | N | N | S | CH | S | H |
| 040 | F₃C | H₃C | S | N | N | S | CH | S | CH₃ |
| 041 | F₃C | H₃C | S | N | N | S | N | O | H |
| 042 | F₃C | H₃C | S | N | N | S | N | O | CH₃ |
| 043 | F₃C | H₃C | S | N | N | S | N | S | H |
| 044 | F₃C | H₃C | S | N | N | S | N | S | CH₃ |
| 045 | F₃C | H₃C | S | N | N | O | CH | O | H |
| 046 | F₃C | H₃C | S | N | N | O | CH | O | CH₃ |
| 047 | F₃C | H₃C | S | N | N | O | CH | S | H |
| 048 | F₃C | H₃C | S | N | N | O | CH | S | CH₃ |
| 049 | H₃C | H₃C | O | CH | N | S | CH | O | H |
| 050 | H₃C | H₃C | O | CH | N | S | CH | O | CH₃ |
| 051 | H₃C | H₃C | O | CH | N | S | CH | S | H |
| 052 | H₃C | H₃C | O | CH | N | S | CH | S | CH₃ |
| 053 | H₃C | H₃C | O | CH | N | S | N | O | H |
| 054 | H₃C | H₃C | O | CH | N | S | N | O | CH₃ |
| 055 | H₃C | H₃C | O | CH | N | S | N | S | H |
| 056 | H₃C | H₃C | O | CH | N | S | N | S | CH₃ |
| 057 | H₃C | H₃C | O | CH | N | O | CH | O | H |
| 058 | H₃C | H₃C | O | CH | N | O | CH | O | CH₃ |
| 059 | H₃C | H₃C | O | CH | N | O | CH | S | H |
| 060 | H₃C | H₃C | O | CH | N | O | CH | S | CH₃ |
| 061 | H₃C | H₃C | O | N | N | S | CH | O | H |
| 062 | H₃C | H₃C | O | N | N | S | CH | O | CH₃ |
| 063 | H₃C | H₃C | O | N | N | S | CH | S | H |
| 064 | H₃C | H₃C | O | N | N | S | CH | S | CH₃ |
| 065 | H₃C | H₃C | O | N | N | S | N | O | H |
| 066 | H₃C | H₃C | O | N | N | S | N | O | CH₃ |
| 067 | H₃C | H₃C | O | N | N | S | N | S | H |
| 068 | H₃C | H₃C | O | N | N | S | N | S | CH₃ |
| 069 | H₃C | H₃C | O | N | N | O | CH | O | H |
| 070 | H₃C | H₃C | O | N | N | O | CH | O | CH₃ |
| 071 | H₃C | H₃C | O | N | N | O | CH | S | H |
| 072 | H₃C | H₃C | O | N | N | O | CH | S | CH₃ |
| 073 | H₃C | H₃C | S | CH | N | S | CH | O | H |
| 074 | H₃C | H₃C | S | CH | N | S | CH | O | CH₃ |
| 075 | H₃C | H₃C | S | CH | N | S | CH | S | H |
| 076 | H₃C | H₃C | S | CH | N | S | CH | S | CH₃ |
| 077 | H₃C | H₃C | S | CH | N | S | N | O | H |
| 078 | H₃C | H₃C | S | CH | N | S | N | O | CH₃ |
| 079 | H₃C | H₃C | S | CH | N | S | N | S | H |
| 080 | H₃C | H₃C | S | CH | N | S | N | S | CH₃ |
| 081 | H₃C | H₃C | S | CH | N | O | CH | O | H |
| 082 | H₃C | H₃C | S | CH | N | O | CH | O | CH₃ |
| 083 | H₃C | H₃C | S | CH | N | O | CH | S | H |
| 084 | H₃C | H₃C | S | CH | N | O | CH | S | CH₃ |
| 085 | H₃C | H₃C | S | N | N | S | CH | O | H |
| 086 | H₃C | H₃C | S | N | N | S | CH | O | CH₃ |
| 087 | H₃C | H₃C | S | N | N | S | CH | S | H |
| 088 | H₃C | H₃C | S | N | N | S | CH | S | CH₃ |
| 089 | H₃C | H₃C | S | N | N | S | N | O | H |
| 090 | H₃C | H₃C | S | N | N | S | N | O | CH₃ |
| 091 | H₃C | H₃C | S | N | N | S | N | S | H |
| 092 | H₃C | H₃C | S | N | N | S | N | S | CH₃ |
| 093 | H₃C | H₃C | S | N | N | O | CH | O | H |
| 094 | H₃C | H₃C | S | N | N | O | CH | O | CH₃ |
| 095 | H₃C | H₃C | S | N | N | O | CH | S | H |
| 096 | H₃C | H₃C | S | N | N | O | CH | S | CH₃ |
| 097 | Cl | H₃C | O | CH | N | S | CH | O | H |
| 098 | Cl | H₃C | O | CH | N | S | CH | O | CH₃ |
| 099 | Cl | H₃C | O | CH | N | S | CH | S | H |
| 100 | Cl | H₃C | O | CH | N | S | CH | S | CH₃ |
| 101 | Cl | H₃C | O | CH | N | S | N | O | H |
| 102 | Cl | H₃C | O | CH | N | S | N | O | CH₃ |
| 103 | Cl | H₃C | O | CH | N | S | N | S | H |
| 104 | Cl | H₃C | O | CH | N | S | N | S | CH₃ |
| 105 | Cl | H₃C | O | CH | N | O | CH | O | H |
| 106 | Cl | H₃C | O | CH | N | O | CH | O | CH₃ |
| 107 | Cl | H₃C | O | CH | N | O | CH | S | H |
| 108 | Cl | H₃C | O | CH | N | O | CH | S | CH₃ |
| 109 | Cl | H₃C | O | N | N | S | CH | O | H |
| 110 | Cl | H₃C | O | N | N | S | CH | O | CH₃ |
| 111 | Cl | H₃C | O | N | N | S | CH | S | H |
| 112 | Cl | H₃C | O | N | N | S | CH | S | CH₃ |
| 113 | Cl | H₃C | O | N | N | S | N | O | H |
| 114 | Cl | H₃C | O | N | N | S | N | O | CH₃ |
| 115 | Cl | H₃C | O | N | N | S | N | S | H |
| 116 | Cl | H₃C | O | N | N | S | N | S | CH₃ |
| 117 | Cl | H₃C | O | N | N | O | CH | O | H |
| 118 | Cl | H₃C | O | N | N | O | CH | O | CH₃ |
| 119 | Cl | H₃C | O | N | N | O | CH | S | H |
| 120 | Cl | H₃C | O | N | N | O | CH | S | CH₃ |
| 121 | Cl | H₃C | S | CH | N | S | CH | O | H |
| 122 | Cl | H₃C | S | CH | N | S | CH | O | CH₃ |
| 123 | Cl | H₃C | S | CH | N | S | CH | S | H |
| 124 | Cl | H₃C | S | CH | N | S | CH | S | CH₃ |
| 125 | Cl | H₃C | S | CH | N | S | N | O | H |
| 126 | Cl | H₃C | S | CH | N | S | N | O | CH₃ |
| 127 | Cl | H₃C | S | CH | N | S | N | S | H |
| 128 | Cl | H₃C | S | CH | N | S | N | S | CH₃ |
| 129 | Cl | H₃C | S | CH | N | O | CH | O | H |
| 130 | Cl | H₃C | S | CH | N | O | CH | O | CH₃ |
| 131 | Cl | H₃C | S | CH | N | O | CH | S | H |
| 132 | Cl | H₃C | S | CH | N | O | CH | S | CH₃ |
| 133 | Cl | H₃C | S | N | N | S | CH | O | H |
| 134 | Cl | H₃C | S | N | N | S | CH | O | CH₃ |
| 135 | Cl | H₃C | S | N | N | S | CH | S | H |
| 136 | Cl | H₃C | S | N | N | S | CH | S | CH₃ |
| 137 | Cl | H₃C | S | N | N | S | N | O | H |
| 138 | Cl | H₃C | S | N | N | S | N | O | CH₃ |
| 139 | Cl | H₃C | S | N | N | S | N | S | H |
| 140 | Cl | H₃C | S | N | N | S | N | S | CH₃ |
| 141 | Cl | H₃C | S | N | N | O | CH | O | H |
| 142 | Cl | H₃C | S | N | N | O | CH | O | CH₃ |
| 143 | Cl | H₃C | S | N | N | O | CH | S | H |
| 144 | Cl | H₃C | S | N | N | O | CH | S | CH₃ |
| 145 | F₂HC | H₃C | O | CH | N | S | CH | O | H |
| 146 | F₂HC | H₃C | O | CH | N | S | CH | O | CH₃ |
| 147 | F₂HC | H₃C | O | CH | N | S | CH | S | H |
| 148 | F₂HC | H₃C | O | CH | N | S | CH | S | CH₃ |
| 149 | F₂HC | H₃C | O | CH | N | S | N | O | H |
| 150 | F₂HC | H₃C | O | CH | N | S | N | O | CH₃ |
| 151 | F₂HC | H₃C | O | CH | N | S | N | S | H |
| 152 | F₂HC | H₃C | O | CH | N | S | N | S | CH₃ |
| 153 | F₂HC | H₃C | O | CH | N | O | CH | O | H |
| 154 | F₂HC | H₃C | O | CH | N | O | CH | O | CH₃ |
| 155 | F₂HC | H₃C | O | CH | N | O | CH | S | H |
| 156 | F₂HC | H₃C | O | CH | N | O | CH | S | CH₃ |
| 157 | F₂HC | H₃C | O | N | N | S | CH | O | H |
| 158 | F₂HC | H₃C | O | N | N | S | CH | O | CH₃ |
| 159 | F₂HC | H₃C | O | N | N | S | CH | S | H |
| 160 | F₂HC | H₃C | O | N | N | S | CH | S | CH₃ |
| 161 | F₂HC | H₃C | O | N | N | S | N | O | H |
| 162 | F₂HC | H₃C | O | N | N | S | N | O | CH₃ |
| 163 | F₂HC | H₃C | O | N | N | S | N | S | H |
| 164 | F₂HC | H₃C | O | N | N | S | N | S | CH₃ |
| 165 | F₂HC | H₃C | O | N | N | O | CH | O | H |
| 166 | F₂HC | H₃C | O | N | N | O | CH | O | CH₃ |
| 167 | F₂HC | H₃C | O | N | N | O | CH | S | H |
| 168 | F₂HC | H₃C | O | N | N | O | CH | S | CH₃ |
| 169 | F₂HC | H₃C | S | CH | N | S | CH | O | H |
| 170 | F₂HC | H₃C | S | CH | N | S | CH | O | CH₃ |
| 171 | F₂HC | H₃C | S | CH | N | S | CH | S | H |
| 172 | F₂HC | H₃C | S | CH | N | S | CH | S | CH₃ |
| 173 | F₂HC | H₃C | S | CH | N | S | N | O | H |
| 174 | F₂HC | H₃C | S | CH | N | S | N | O | CH₃ |
| 175 | F₂HC | H₃C | S | CH | N | S | N | S | H |
| 176 | F₂HC | H₃C | S | CH | N | S | N | S | CH₃ |
| 177 | F₂HC | H₃C | S | CH | N | O | CH | O | H |
| 178 | F₂HC | H₃C | S | CH | N | O | CH | O | CH₃ |
| 179 | F₂HC | H₃C | S | CH | N | O | CH | S | H |
| 180 | F₂HC | H₃C | S | CH | N | O | CH | S | CH₃ |
| 181 | F₂HC | H₃C | S | N | N | S | CH | O | H |
| 182 | F₂HC | H₃C | S | N | N | S | CH | O | CH₃ |
| 183 | F₂HC | H₃C | S | N | N | S | CH | S | H |
| 184 | F₂HC | H₃C | S | N | N | S | CH | S | CH₃ |
| 185 | F₂HC | H₃C | S | N | N | S | N | O | H |
| 186 | F₂HC | H₃C | S | N | N | S | N | O | CH₃ |
| 187 | F₂HC | H₃C | S | N | N | S | N | S | H |
| 188 | F₂HC | H₃C | S | N | N | S | N | S | CH₃ |
| 189 | F₂HC | H₃C | S | N | N | O | CH | O | H |
| 190 | F₂HC | H₃C | S | N | N | O | CH | O | CH₃ |
| 191 | F₂HC | H₃C | S | N | N | O | CH | S | H |
| 192 | F₂HC | H₃C | S | N | N | O | CH | S | CH₃ |
| 193 | F₂HC | F₂HC | O | CH | N | S | CH | O | H |
| 194 | F₂HC | F₂HC | O | CH | N | S | CH | O | CH3 |
| 195 | F₂HC | F₂HC | O | CH | N | S | CH | S | H |
| 196 | F₂HC | F₂HC | O | CH | N | S | CH | S | CH3 |
| 197 | F₂HC | F₂HC | O | CH | N | S | N | O | H |
| 198 | F₂HC | F₂HC | O | CH | N | S | N | O | CH3 |
| 199 | F₂HC | F₂HC | O | CH | N | S | N | S | H |
| 200 | F₂HC | F₂HC | O | CH | N | S | N | S | CH3 |
| 201 | F₂HC | F₂HC | O | CH | N | O | CH | O | H |
| 202 | F₂HC | F₂HC | O | CH | N | O | CH | O | CH3 |
| 203 | F₂HC | F₂HC | O | CH | N | O | CH | S | H |
| 204 | F₂HC | F₂HC | O | CH | N | O | CH | S | CH3 |
| 205 | F₂HC | F₂HC | O | N | N | S | CH | O | H |
| 206 | F₂HC | F₂HC | O | N | N | S | CH | O | CH3 |
| 207 | F₂HC | F₂HC | O | N | N | S | CH | S | H |
| 208 | F₂HC | F₂HC | O | N | N | S | CH | S | CH3 |
| 209 | F₂HC | F₂HC | O | N | N | S | N | O | H |
| 210 | F₂HC | F₂HC | O | N | N | S | N | O | CH3 |
| 211 | F₂HC | F₂HC | O | N | N | S | N | S | H |
| 212 | F₂HC | F₂HC | O | N | N | S | N | S | CH3 |
| 213 | F₂HC | F₂HC | O | N | N | O | CH | O | H |
| 214 | F₂HC | F₂HC | O | N | N | O | CH | O | CH3 |
| 215 | F₂HC | F₂HC | O | N | N | O | CH | S | H |
| 216 | F₂HC | F₂HC | O | N | N | O | CH | S | CH3 |
| 217 | F₂HC | F₂HC | S | CH | N | S | CH | O | H |
| 218 | F₂HC | F₂HC | S | CH | N | S | CH | O | CH3 |
| 219 | F₂HC | F₂HC | S | CH | N | S | CH | S | H |
| 220 | F₂HC | F₂HC | S | CH | N | S | CH | S | CH3 |
| 221 | F₂HC | F₂HC | S | CH | N | S | N | O | H |
| 222 | F₂HC | F₂HC | S | CH | N | S | N | O | CH3 |
| 223 | F₂HC | F₂HC | S | CH | N | S | N | S | H |
| 224 | F₂HC | F₂HC | S | CH | N | S | N | S | CH3 |
| 225 | F₂HC | F₂HC | S | CH | N | O | CH | O | H |
| 226 | F₂HC | F₂HC | S | CH | N | O | CH | O | CH3 |
| 227 | F₂HC | F₂HC | S | CH | N | O | CH | S | H |
| 228 | F₂HC | F₂HC | S | CH | N | O | CH | S | CH3 |
| 229 | F₂HC | F₂HC | S | N | N | S | CH | O | H |
| 230 | F₂HC | F₂HC | S | N | N | S | CH | O | CH3 |
| 231 | F₂HC | F₂HC | S | N | N | S | CH | S | H |
| 232 | F₂HC | F₂HC | S | N | N | S | CH | S | CH3 |
| 233 | F₂HC | F₂HC | S | N | N | S | N | O | H |
| 234 | F₂HC | F₂HC | S | N | N | S | N | O | CH3 |
| 235 | F₂HC | F₂HC | S | N | N | S | N | S | H |
| 236 | F₂HC | F₂HC | S | N | N | S | N | S | CH3 |
| 237 | F₂HC | F₂HC | S | N | N | O | CH | O | H |
| 238 | F₂HC | F₂HC | S | N | N | O | CH | O | CH3 |
| 239 | F₂HC | F₂HC | S | N | N | O | CH | S | H |
| 240 | F₂HC | F₂HC | S | N | N | O | CH | S | CH3 |

where
a) 240 compounds of formula (I.a):

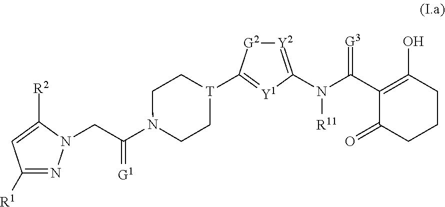

wherein R¹, R², R¹¹, G¹, G², G³, T, Y¹ and Y² are as defined in Table 1.
b) 240 compounds of formula (I.b):

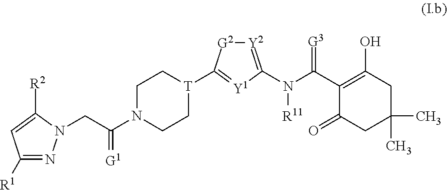

wherein R¹, R², R¹¹, G¹, G², G³, T, Y¹ and Y² are as defined in Table 1.
c) 240 compounds of formula (I.c):

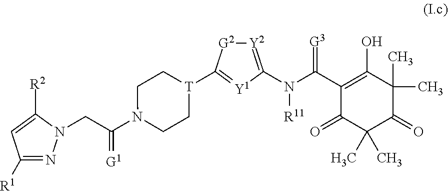

wherein R¹, R², R¹¹, G¹, G², G³, T, Y¹ and Y² are as defined in Table 1.
d) 240 compounds of formula (I.d):

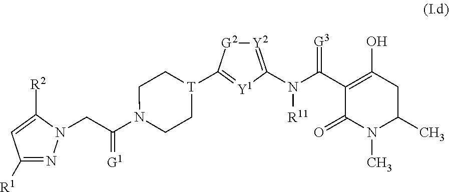

wherein R¹, R², R¹¹, G¹, G², G³, T, Y¹ and Y² are as defined in Table 1.
e) 240 compounds of formula (Ie):

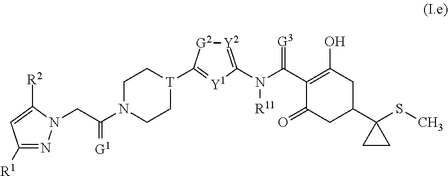

wherein R¹, R², R¹¹, G¹, G², G³, T, Y¹ and Y² are as defined in Table 1.
f) 240 compounds of formula (I.f):

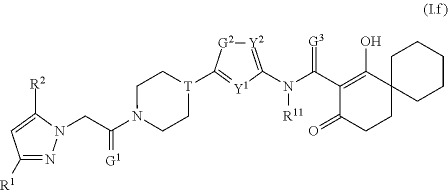

wherein R¹, R², R¹¹, G¹, G², G³, T, Y¹ and Y² are as defined in Table 1.
g) 240 compounds of formula (I.g):

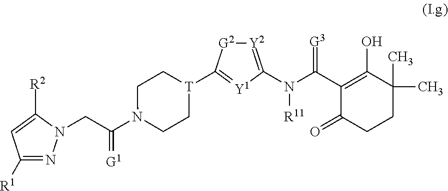

wherein R¹, R², R¹¹, G¹, G², G³, T, Y¹ and Y² are as defined in Table 1.
h) 240 compounds of formula (I.h):

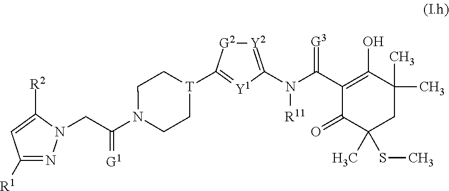

wherein R¹, R², R¹¹, G¹, G², G³, T, Y¹ and Y² are as defined in Table 1.
i) 240 compounds of formula (I.i):

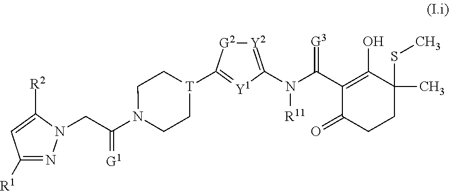

wherein R¹, R², R¹¹, G¹, G², G³, T, Y¹ and Y² are as defined in Table 1.
j) 240 compounds of formula (I.j):

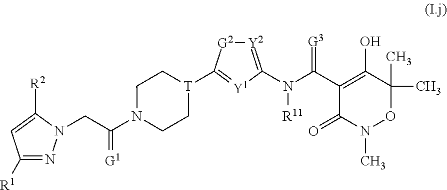

wherein R¹, R², R¹¹, G¹, G², G³, T, Y¹ and Y² are as defined in Table 1.
k) 240 compounds of formula (I.k):

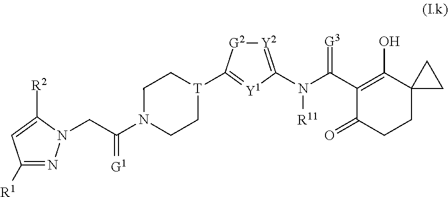

wherein R¹, R², R¹¹, G¹, G², G³, T, Y¹ and Y² are as defined in Table 1.
m) 240 compounds of formula (I.m):

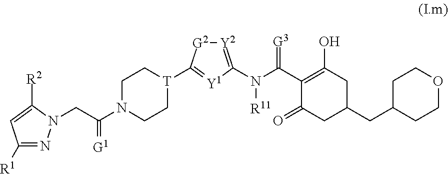

wherein R¹, R², R¹¹, G¹, G², G³, T, Y¹ and Y² are as defined in Table 1.
n) 240 compounds of formula (I.n):

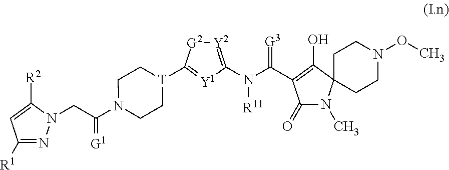

wherein R¹, R², R¹¹, G¹, G², G³, T, Y¹ and Y² are as defined in Table 1.
o) 240 compounds of formula (I.o):

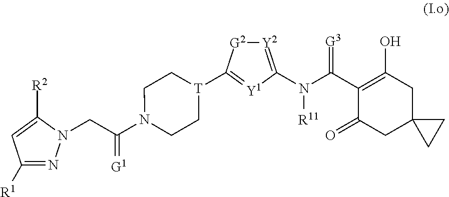

wherein R¹, R², R¹¹, G¹, G², G³, T, Y¹ and Y² are as defined in Table 1.
p) 240 compounds of formula (I.p):

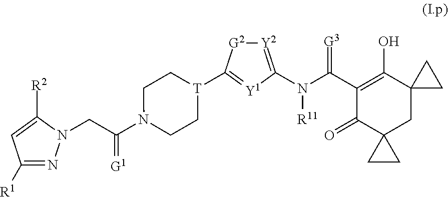

wherein R¹, R², R¹¹, G¹, G², G³, T, Y¹ and Y² are as defined in Table 1.
q) 240 compounds of formula (I.q):

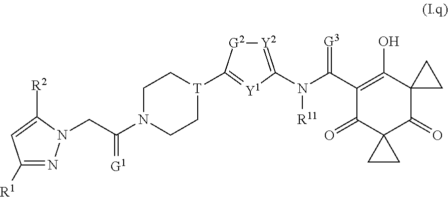

wherein R¹, R², R¹¹, G¹, G², G³, T, Y¹ and Y² are as defined in Table 1.
r) 240 compounds of formula (I.r):

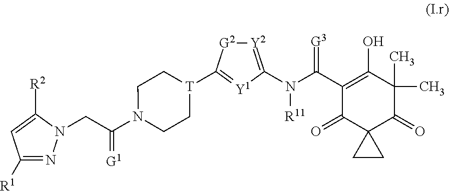

wherein R¹, R², R¹¹, G¹, G², G³, T, Y¹ and Y² are as defined in Table 1.
s) 240 compounds of formula (I.s):

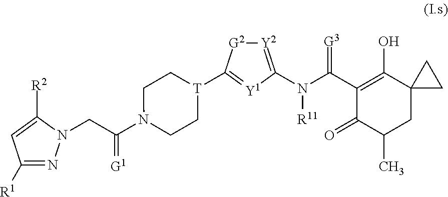

wherein R¹, R², R¹¹, G¹, G², G³, T, Y¹ and Y² are as defined in Table 1.
t) 240 compounds of formula (I.t):

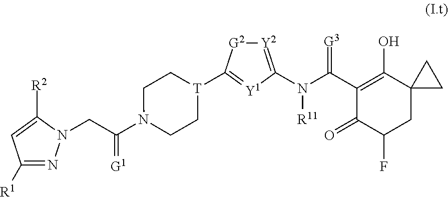

wherein R¹, R², R¹¹, G¹, G², G³, T, Y¹ and Y² are as defined in Table 1.
u) 240 compounds of formula (I.u):

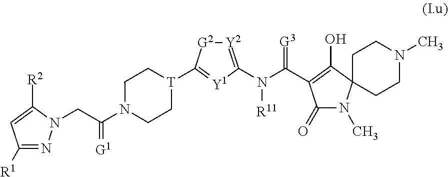

wherein R¹, R², R¹¹, G¹, G², G³, T, Y¹ and Y² are as defined in Table 1.
v) 240 compounds of formula (I.v):

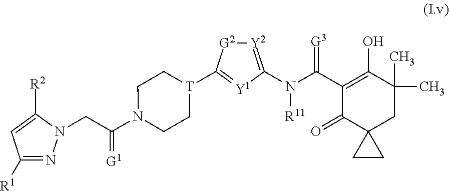

wherein R¹, R², R¹¹, G¹, G², G³, T, Y¹ and Y² are as defined in Table 1.
w) 240 compounds of formula (I.w):

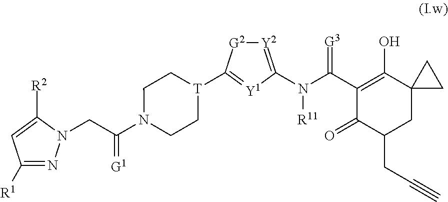

wherein R¹, R², R¹¹, G¹, G², G³, T, Y¹ and Y² are as defined in Table 1.
x) 240 compounds of formula (I.x):

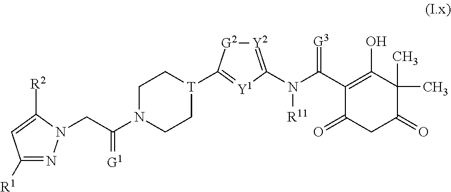

wherein R¹, R², R¹¹, G¹, G², G³, T, Y¹ and Y² are as defined in Table 1.
y) 240 compounds of formula (I.y):

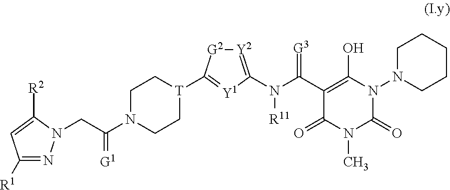

wherein R¹, R², R¹¹, G¹, G², G³, T, Y¹ and Y² are as defined in Table 1.
z) 240 compounds of formula (I.z):

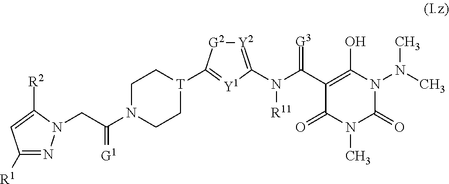

wherein R¹, R², R¹¹, G¹, G², G³, T, Y¹ and Y² are as defined in Table 1.
aa) 240 compounds of formula (I.aa):

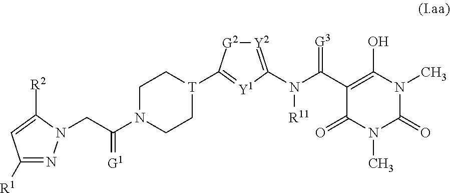

wherein R¹, R², R¹¹, G¹, G², G³, T, Y¹ and Y² are as defined in Table 1.
ab) 240 compounds of formula (I.ab):

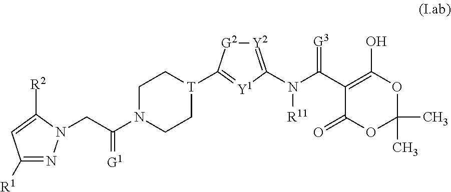

wherein R¹, R², R¹¹, G¹, G², G³, T, Y¹ and Y² are as defined in Table 1.
ac) 240 compounds of formula (I.ac):

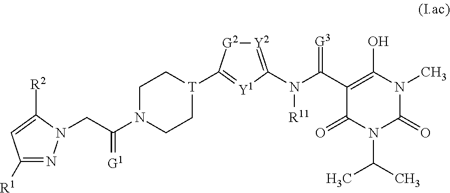

wherein R¹, R², R¹¹, G¹, G², G³, T, Y¹ and Y² are as defined in Table 1.
ad) 240 compounds of formula (I.ad):

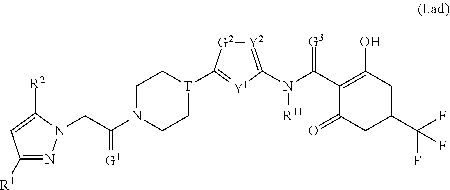

wherein R¹, R², R¹¹, G¹, G², G³, T, Y¹ and Y² are as defined in Table 1.
ae) 240 compounds of formula (I.ae):

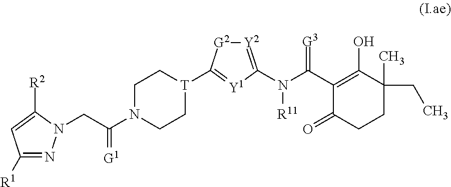

wherein R¹, R², R¹¹, G¹, G², G³, T, Y¹ and Y² are as defined in Table 1.
af) 240 compounds of formula (I.af):

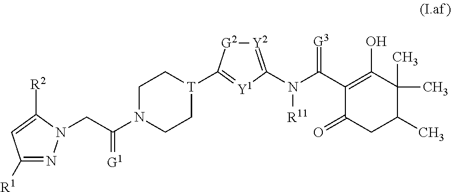

wherein R¹, R², R¹¹, G¹, G², G³, T, Y¹ and Y² are as defined in Table 1.
ag) 240 compounds of formula (I.ag):

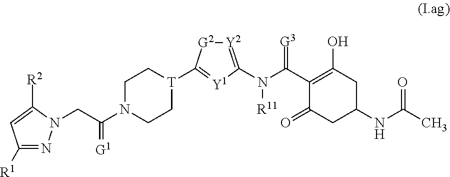

wherein R¹, R², R¹¹, G¹, G², G³, T, Y¹ and Y² are as defined in Table 1.
ah) 240 compounds of formula (I.ah):

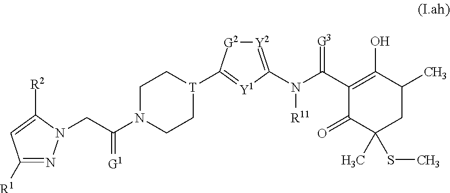

wherein R¹, R², R¹¹, G¹, G², G³, T, Y¹ and Y² are as defined in Table 1.
ai) 240 compounds of formula (I.ai):

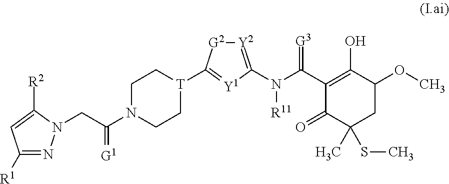

wherein R¹, R², R¹¹, G¹, G², G³, T, Y¹ and Y² are as defined in Table 1.
aj) 192 compounds of formula (I.aj):

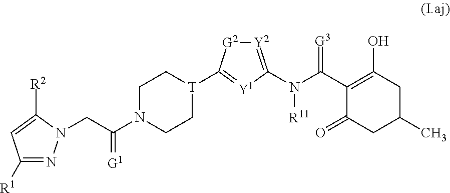

wherein R¹, R², R¹¹, G¹, G², G³, T, Y¹ and Y² are as defined in Table 1.
ak) 240 compounds of formula (I.ak):

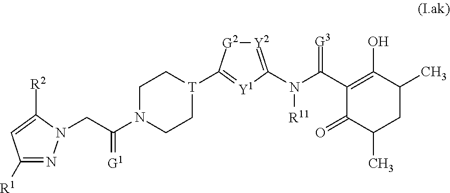

wherein R¹, R², R¹¹, G¹, G², G³, T, Y¹ and Y² are as defined in Table 1.
am) 240 compounds of formula (I.am):

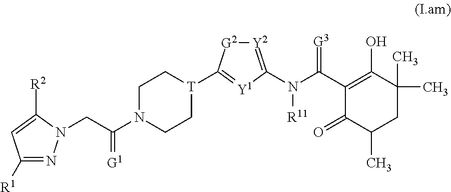

wherein R¹, R², R¹¹, G¹, G², G³, T, Y¹ and Y² are as defined in Table 1.
an) 240 compounds of formula (I.an):

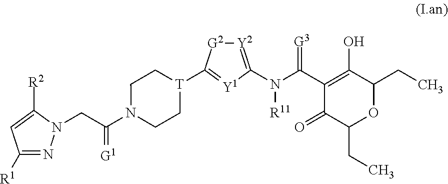

wherein R¹, R², R¹¹, G¹, G², G³, T, Y¹ and Y² are as defined in Table 1.
ao) 240 compounds of formula (I.ao):

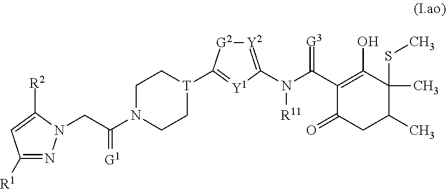

wherein R¹, R², R¹¹, G¹, G², G³, T, Y¹ and Y² are as defined in Table 1.
ap) 240 compounds of formula (I.ap):

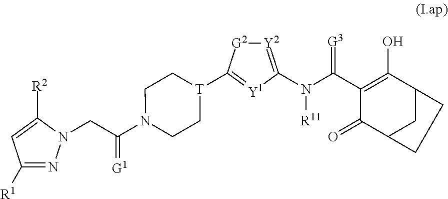

wherein R¹, R², R¹¹, G¹, G², G³, T, Y¹ and Y² are as defined in Table 1.
aq) 240 compounds of formula (I.aq):

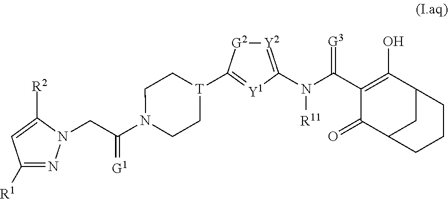

wherein R¹, R², R¹¹, G¹, G², G³, T, Y¹ and Y² are as defined in Table 1.
ar) 240 compounds of formula (I.ar):

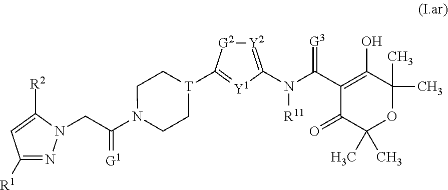

wherein R¹, R², R¹¹, G¹, G², G³, T, Y¹ and Y² are as defined in Table 1.
as) 192 compounds of formula (I.as):

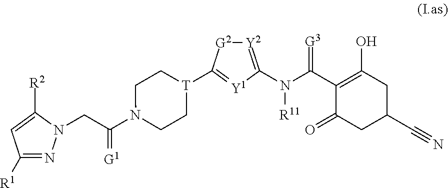

wherein R¹, R², R¹¹, G¹, G², G³, T, Y¹ and Y² are as defined in Table 1.
at) 240 compounds of formula (I.at):

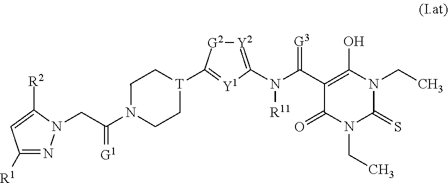

wherein R¹, R², R¹¹, G¹, G², G³, T, Y¹ and Y² are as defined in Table 1.
au) 240 compounds of formula (I.au):

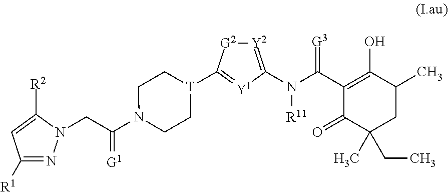

wherein R¹, R², R¹¹, G¹, G², G³, T, Y¹ and Y² are as defined in Table 1.
av) 240 compounds of formula (I.av):

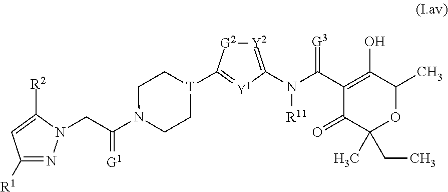

wherein R¹, R², R¹¹, G¹, G², G³, T, Y¹ and Y² are as defined in Table 1.
aw) 240 compounds of formula (I.aw):

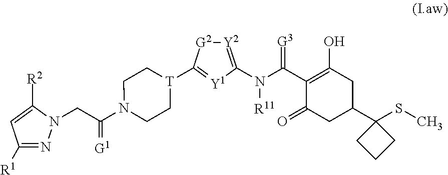

wherein R¹, R², R¹¹, G¹, G², G³, T, Y¹ and Y² are as defined in Table 1.
ax) 240 compounds of formula (I.ax):

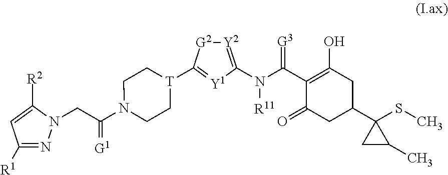

wherein R¹, R², R¹¹, G¹, G², G³, T, Y¹ and Y² are as defined in Table 1.
ay) 240 compounds of formula (I.ay):

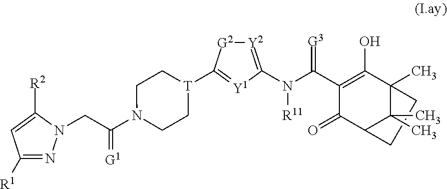

wherein R¹, R², R¹¹, G¹, G², G³, T, Y¹ and Y² are as defined in Table 1.
az) 192 compounds of formula (I.az):

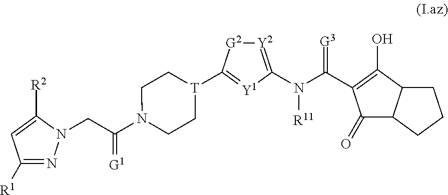

wherein R¹, R², R¹¹, G¹, G², G³, T, Y¹ and Y² are as defined in Table 1.
ba) 240 compounds of formula (I.ba):

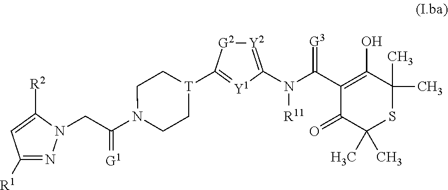

wherein R¹, R², R¹¹, G¹, G², G³, T, Y¹ and Y² are as defined in Table 1.
bb) 240 compounds of formula (I.bb):

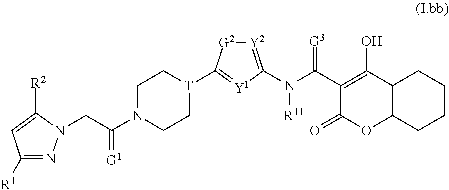

wherein R¹, R², R¹¹, G¹, G², G³, T, Y¹ and Y² are as defined in Table 1.
bc) 240 compounds of formula (I.bc):

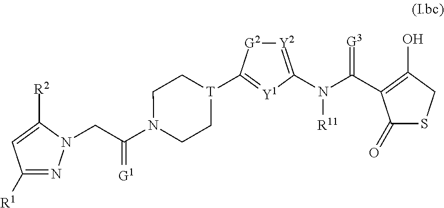

wherein R¹, R², R¹¹, G¹, G², G³, T, Y¹ and Y² are as defined in Table 1.
bd) 240 compounds of formula (I.bd):

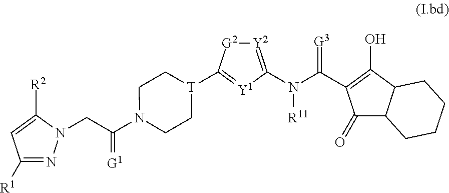

wherein R¹, R², R¹¹, G¹, G², G³, T, Y¹ and Y² are as defined in Table 1.
be) 240 compounds of formula (I.be):

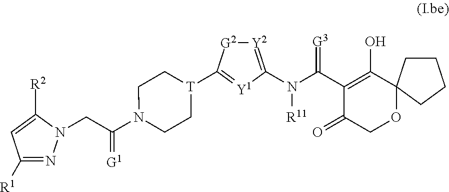

wherein R¹, R², R¹¹, G¹, G², G³, T, Y¹ and Y² are as defined in Table 1.
bf) 240 compounds of formula (I.bf):

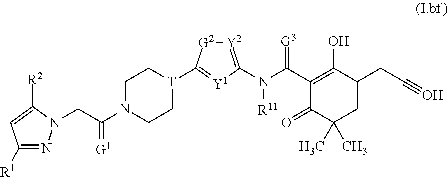

wherein R¹, R², R¹¹, G¹, G², G³, T, Y¹ and Y² are as defined in Table 1.
bg) 240 compounds of formula (I.bg):

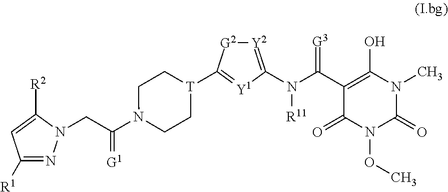

wherein R¹, R², R¹¹, G¹, G², G³, T, Y¹ and Y² are as defined in Table 1.
bh) 240 compounds of formula (I.bh):

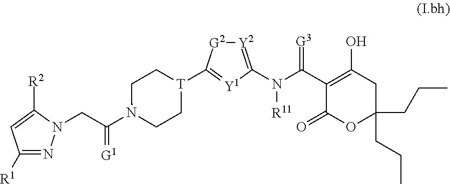

wherein R¹, R², R¹¹, G¹, G², G³, T, Y¹ and Y² are as defined in Table 1.
bi) 240 compounds of formula (I.bi):

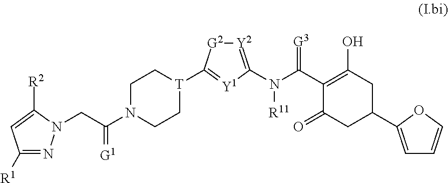

wherein R¹, R², R¹¹, G¹, G², G³, T, Y¹ and Y² are as defined in Table 1.
bj) 240 compounds of formula (I.bj):

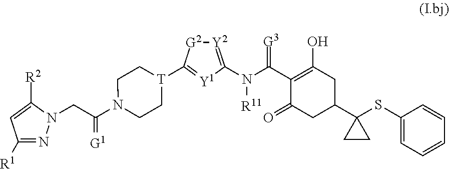

wherein R¹, R², R¹¹, G¹, G², G³, T, Y¹ and Y² are as defined in Table 1.
bk) 240 compounds of formula (I.bk):

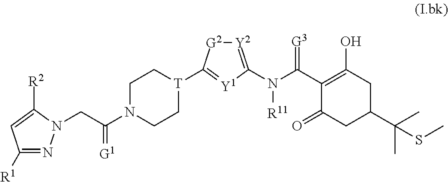

wherein R¹, R², R¹¹, G¹, G², G³, T, Y¹ and Y² are as defined in Table 1.
bm) 240 compounds of formula (I.bm):

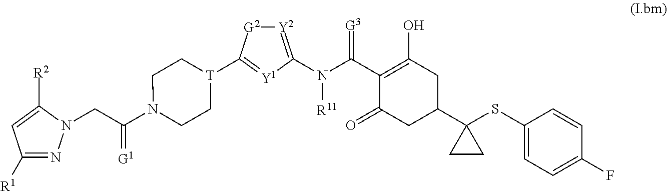

wherein R¹, R², R¹¹, G¹, G², G³, T, Y¹ and Y² are as defined in Table 1.
bn) 240 compounds of formula (I.bn):

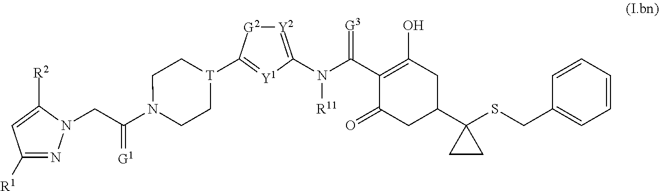

wherein R¹, R², R¹¹, G¹, G², G³, T, Y¹ and Y² are as defined in Table 1.
bo) 240 compounds of formula (I.bo):

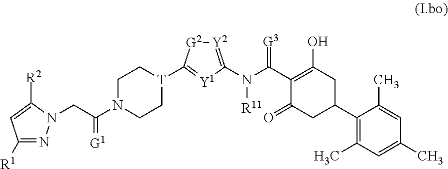

wherein R¹, R², R¹¹, G¹, G², G³, T, Y¹ and Y² are as defined in Table 1.
bp) 240 compounds of formula (I.bp):

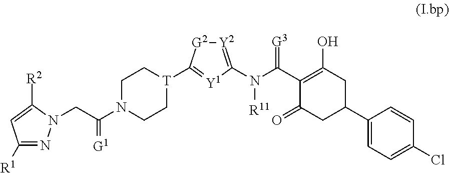

wherein R¹, R², R¹¹, G¹, G², G³, T, Y¹ and Y² are as defined in Table 1.
bq) 240 compounds of formula (I.bq):

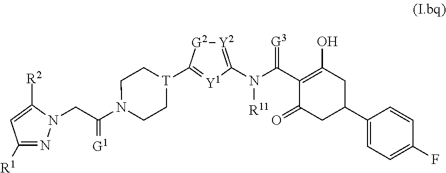

wherein R¹, R², R¹¹, G¹, G², G³, T, Y¹ and Y² are as defined in Table 1.
br) 240 compounds of formula (I.br):

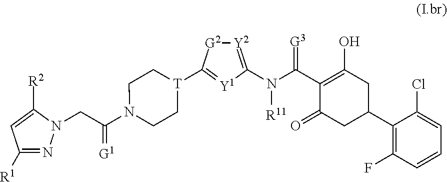

wherein R¹, R², R¹¹, G¹, G², G³, T, Y¹ and Y² are as defined in Table 1.
bs) 240 compounds of formula (I.bs):

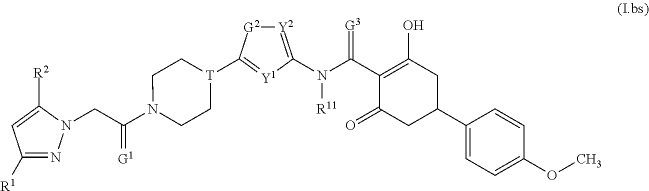

wherein R¹, R², R¹¹, G¹, G², G³, T, Y¹ and Y² are as defined in Table 1.
bt) 240 compounds of formula (I.bt):

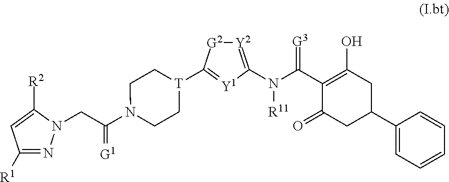

wherein R¹, R², R¹¹, G¹, G², G³, T, Y¹ and Y² are as defined in Table 1.
bu) 240 compounds of formula (I.bu):

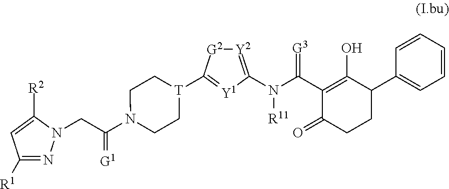

wherein R¹, R², R¹¹, G¹, G², G³, T, Y¹ and Y² are as defined in Table 1.
bv) 240 compounds of formula (I.bv):

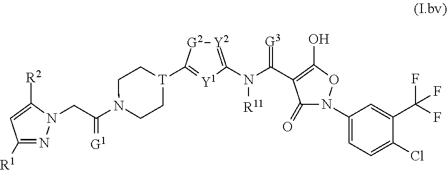

wherein R¹, R², R¹¹, G¹, G², G³, T, Y¹ and Y² are as defined in Table 1.
bw) 240 compounds of formula (I.bw):

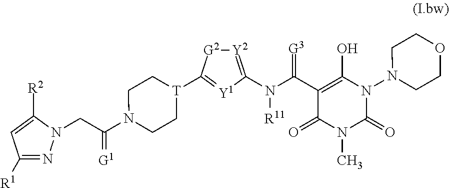

wherein R¹, R², R¹¹, G¹, G², G³, T, Y¹ and Y² are as defined in Table 1.
bx) 240 compounds of formula (I.bx):

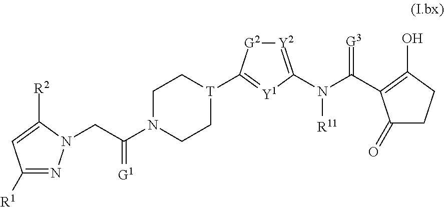

wherein R¹, R², R¹¹, G¹, G², G³, T, Y¹ and Y² are as defined in Table 1.
by) 240 compounds of formula (I.by):

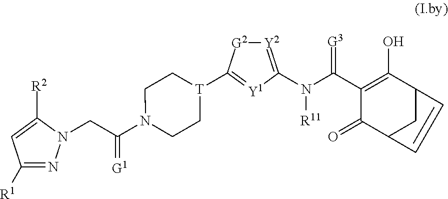

wherein R¹, R², R¹¹, G¹, G², G³, T, Y¹ and Y² are as defined in Table 1.
bz) 240 compounds of formula (I.bz):

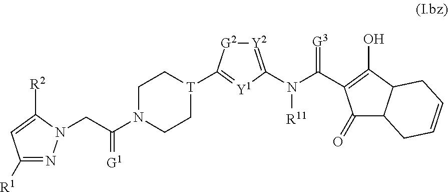

wherein R¹, R², R¹¹, G¹, G², G³, T, Y¹ and Y² are as defined in Table 1.
ca) 240 compounds of formula (I.ca):

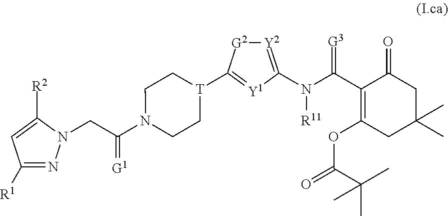

Wherein R¹, R², R¹¹, G¹, G², G³, T, Y¹ and Y² are as defined in Table 1.
cb) 240 compounds of formula (I.cb):

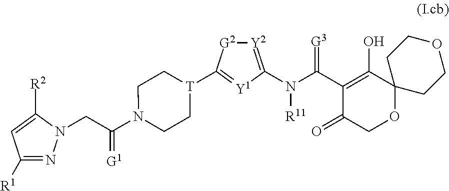

Wherein R¹, R², R¹¹, G¹, G², G³, T, Y¹ and Y² are as defined in Table 1.
cc) 240 compounds of formula (I.cc):

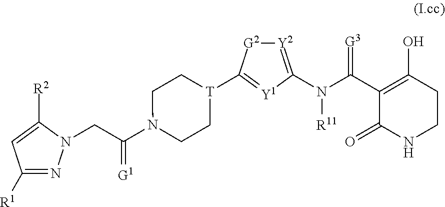

Wherein R¹, R², R¹¹, G¹, G², G³, T, Y¹ and Y² are as defined in Table 1.
cd) 240 compounds of formula (I.cd):

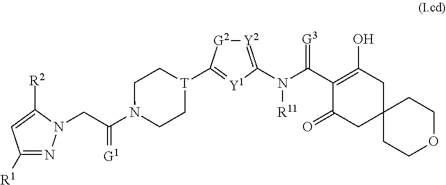

Wherein R¹, R², R¹¹, G¹, G², G³, T, Y¹ and Y² are as defined in Table 1.
ce) 240 compounds of formula (I.ce):

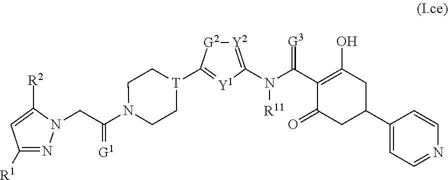

Wherein R¹, R², R¹¹, G¹, G², G³, T, Y¹ and Y² are as defined in Table 1.
cf) 240 compounds of formula (I.cf):

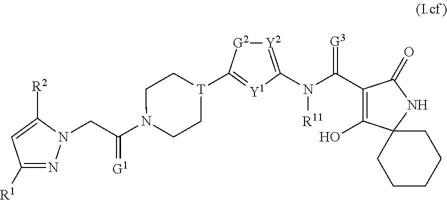

Wherein R¹, R², R¹¹, G¹, G², G³, T, Y¹ and Y² are as defined in Table 1.
cg) 240 compounds of formula (I.cg):

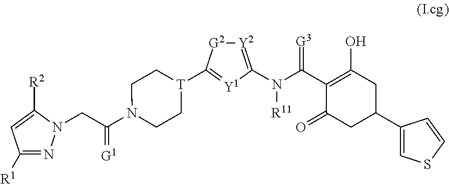

Wherein R¹, R², R¹¹, G¹, G², G³, T, Y¹ and Y² are as defined in Table 1.
ch) 240 compounds of formula (I.ch):

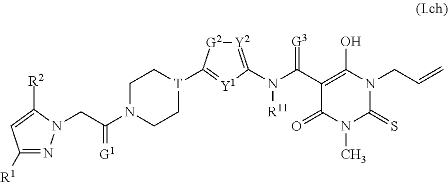

Wherein R¹, R², R¹¹, G¹, G², G³, T, Y¹ and Y² are as defined in Table 1.
ci) 240 compounds of formula (I.ci):

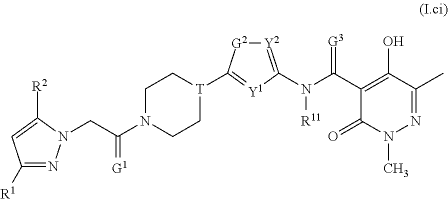

Wherein R¹, R², R¹¹, G¹, G², G³, T, Y¹ and Y² are as defined in Table 1.
cj) 240 compounds of formula (I.cj):

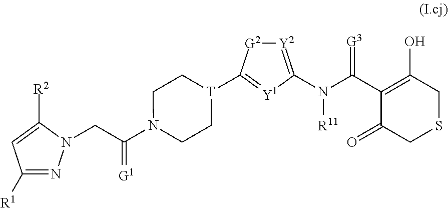

Wherein R¹, R², R¹¹, G¹, G², G³, T, Y¹ and Y² are as defined in Table 1.
ck) 240 compounds of formula (I.ck):

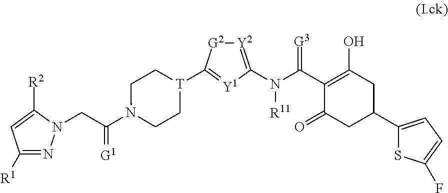

Wherein R¹, R², R¹¹, G¹, G², G³, T, Y¹ and Y² are as defined in Table 1.
cl) 240 compounds of formula (I.cl):

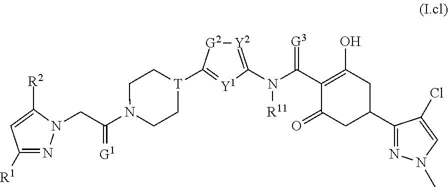

Wherein R¹, R², R¹¹, G¹, G², G³, T, Y¹ and Y² are as defined in Table 1.
cm) 240 compounds of formula (I.cm):

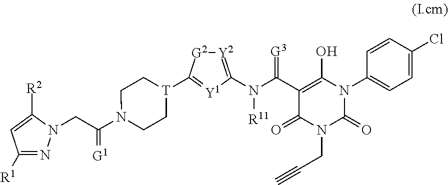

Wherein R¹, R², R¹¹, G¹, G², G³, T, Y¹ and Y² are as defined in Table 1.
cn) 240 compounds of formula (I.cn):

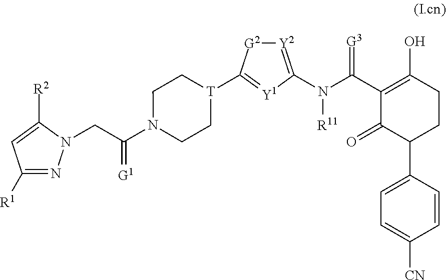

Wherein R¹, R², R¹¹, G¹, G², G³, T, Y¹ and Y² are as defined in Table 1.
co) 240 compounds of formula (I.co):

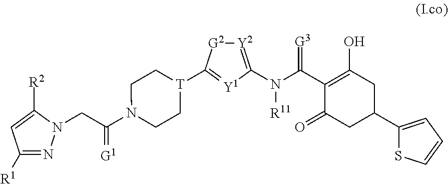

Wherein R¹, R², R¹¹, G¹, G², G³, T, Y¹ and Y² are as defined in Table 1.
cp) 240 compounds of formula (I.cp):

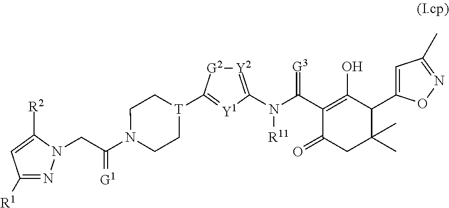

Wherein R¹, R², R¹¹, G¹, G², G³, T, Y¹ and Y² are as defined in Table 1.
cq) 240 compounds of formula (I.cq):

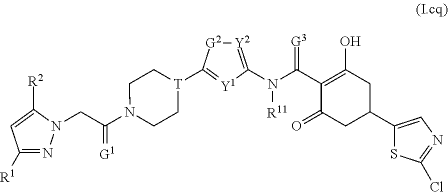

Wherein R¹, R², R¹¹, G¹, G², G³, T, Y¹ and Y² are as defined in Table 1.
cr) 240 compounds of formula (I.cr):

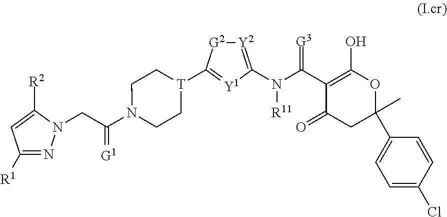

Wherein R¹, R², R¹¹, G¹, G², G³, T, Y¹ and Y² are as defined in Table 1.
cs) 240 compounds of formula (I.cs):

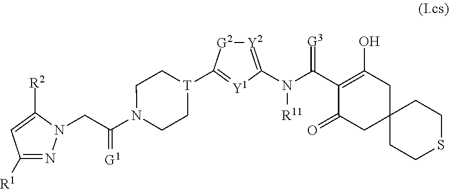

Wherein R¹, R², R¹¹, G¹, G², G³, T, Y¹ and Y² are as defined in Table 1.
ct) 240 compounds of formula (I.ct):

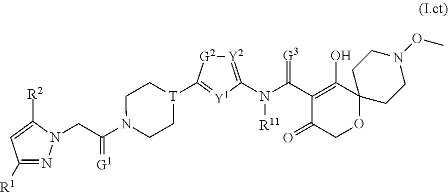

Wherein R¹, R², R¹¹, G¹, G², G³, T, Y¹ and Y² are as defined in Table 1.
cu) 240 compounds of formula (I.cu):

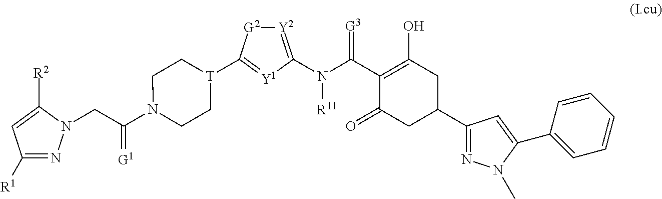

Wherein R¹, R², R¹¹, G¹, G², G³, T, Y¹ and Y² are as defined in Table 1.
cv) 240 compounds of formula (I.cv):

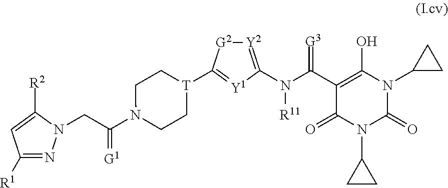

Wherein R¹, R², R¹¹, G¹, G², G³, T, Y¹ and Y² are as defined in Table 1.
cw) 240 compounds of formula (I.cw):

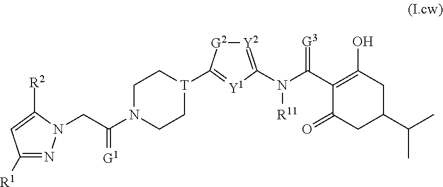

Wherein R¹, R², R¹¹, G¹, G², G³, T, Y¹ and Y² are as defined in Table 1.
cx) 240 compounds of formula (I.cx):

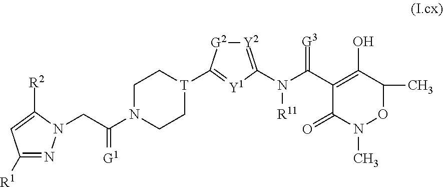

Wherein R¹, R², R¹¹, G¹, G², G³, T, Y¹ and Y² are as defined in Table 1.
cy) 240 compounds of formula (I.cy):

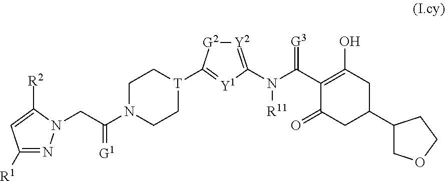

Wherein R¹, R², R¹¹, G¹, G², G³, T, Y¹ and Y² are as defined in Table 1.
cz) 240 compounds of formula (I.cz):

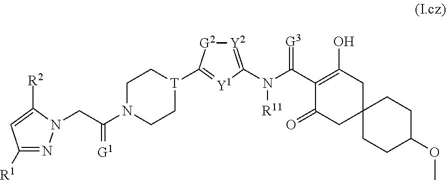

Wherein R¹, R², R¹¹, G¹, G², G³, T, Y¹ and Y² are as defined in Table 1.
da) 240 compounds of formula (I.da):

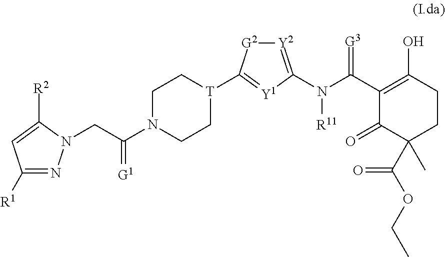

Wherein R¹, R², R¹¹, G¹, G², G³, T, Y¹ and Y² are as defined in Table 1.
db) 240 compounds of formula (I.db):

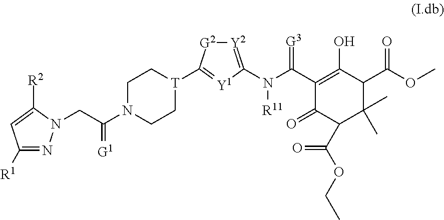

Wherein R¹, R², R¹¹, G¹, G², G³, T, Y¹ and Y² are as defined in Table 1.
dc) 240 compounds of formula (I.dc):

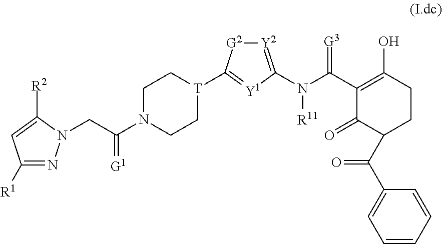

Wherein R¹, R², R¹¹, G¹, G², G³, T, Y¹ and Y² are as defined in Table 1.

Throughout this description, temperatures are given in degrees Celsius and “m.p.” means melting point. LC/MS means Liquid Chromatography Mass Spectroscopy and the description of the apparatus and the method is: (ACQUITY UPLC from Waters, Phenomenex Gemini C18, 3 μm particle size, 110 Angström, 30×3 mm column, 1.7 mL/min., 60° C., H₂O+0.05% HCOOH (95%)/CH₃CN/MeOH 4:1+0.04% HCOOH (5%)—2 min.—CH₃CN/MeOH 4:1+0.04% HCOOH (5%)-0.8 min., ACQUITY SQD Mass Spectrometer from Waters, ionization method: electrospray (ESI), Polarity: positive ions, Capillary (kV) 3.00, Cone (V) 20.00, Extractor (V) 3.00, Source Temperature (° C.) 150, Desolvation Temperature (° C.) 400, Cone Gas Flow (L/Hr) 60, Desolvation Gas Flow (L/Hr) 700)).

TABLE 2
Melting point and LC/MS data for compounds of Table 1

| Compound | Melting | |
|---|---|---|
| No. | point (° C.) | LC/MS |
| I.a.001 | 102-105 | |
| I.b.001 | 97-100 | |
| I.c.001 | 157-162 | |
| I.e.001 | | Rt = 2.03 min; MS: m/z = 598.38 (M + 1) |
| I.f.001 | | Rt = 2.19 min; MS: m/z = 580.37 (M + 1) |
| I.g.001 | | Rt = 1.96 min; MS: m/z = 540.35 (M + 1) |
| I.h.001 | | Rt = 2.21 min; MS: m/z = 600.24 (M + 1) |
| I.i.001 | | Rt = 1.98 min; MS: m/z = 572.34 (M + 1) |
| I.j.001 | | Rt = 1.92 min; MS: m/z = 557.33 (M + 1) |
| I.k.001 | | Rt = 1.91 min; MS: m/z = 538.32 (M + 1) |
| I.m.001 | | Rt = 1.87 min; MS: m/z = 610.41 (M + 1) |
| I.n.001 | 116-120 | |
| I.s.001 | | Rt = 2.04 min; MS: m/z = 552.35 (M + 1) |
| I.v.001 | | Rt = 2.13 min; MS: m/z = 566.36 (M + 1) |
| I.y.001 | | Rt = 1.98 min; MS: m/z = 625.39 (M + 1) |
| I.z.001 | | Rt = 1.68 min; MS: m/z = 585.36 (M + 1) |
| I.aa.001 | | Rt = 1.71 min; MS: m/z = 556.33 (M + 1) |
| I.ab.001 | | Rt = 1.47 min; MS: m/z = 544.31 (M + 1) |
| I.ac.001 | | Rt = 1.97 min; MS: m/z = 584.34 (M + 1) |
| I.ad.001 | | Rt = 1.84 min; MS: m/z = 580.31 (M + 1) |
| I.ae.001 | | Rt = 2.05 min; MS: m/z = 554.36 (M + 1) |
| I.af.001 | | Rt = 2.04 min; MS: m/z = 554.36 (M + 1) |
| I.ag.001 | | Rt = 1.40 min; MS: m/z = 569.33 (M + 1) |
| I.ah.001 | | Rt = 2.13 min; MS: m/z = 586.32 (M + 1) |
| I.ai.001 | | Rt = 1.95 min; MS: m/z = 602.32 (M + 1) |
| I.aj.001 | | Rt = 1.83 min; MS: m/z = 525.69 (M + 1) |
| I.ak.001 | | Rt = 2.01 min; MS: m/z = 539.74 (M + 1) |
| I.am.001 | | Rt = 2.10 min; MS: m/z = 554.37 (M + 1) |
| I.an.001 | | Rt = 2.09 min; MS: m/z = 570.39 (M + 1) |
| I.ao.001 | | Rt = 2.04 min; MS: m/z = 586.37 (M + 1) |
| I.ap.001 | | Rt = 1.82 min; MS: m/z = 538.34 (M + 1) |
| I.aq.001 | | Rt = 1.93 min; MS: m/z = 552.35 (M + 1) |
| I.ar.001 | | Rt = 2.09 min; MS: m/z = 570.38 (M + 1) |
| I.as.001 | | Rt = 1.56 min; MS: m/z = 537.33 (M + 1) |
| I.at.001 | | Rt = 2.22 min; MS: m/z = 600.40 (M + 1) |
| I.au.001 | | Rt = 2.19 min; MS: m/z = 568.38 (M + 1) |
| I.av.001 | | Rt = 2.11 min; MS: m/z = 570.37 (M + 1) |
| I.aw.001 | | Rt = 2.09 min; MS: m/z = 612.36 (M + 1) |
| I.ax.001 | | Rt = 2.14 min; MS: m/z = 612.35 (M + 1) |
| I.ay.001 | | Rt = 2.10 min; MS: m/z = 580.35 (M + 1) |
| I.ba.001 | | Rt = 2.21 min; MS: m/z = 586.36 (M + 1) |
| I.bb.001 | | Rt = 1.94 min; MS: m/z = 568.36 (M + 1) |
| I.bc.001 | | Rt = 1.33 min; MS: m/z = 516.26 (M + 1) |
| I.be.001 | | Rt = 2.01 min; MS: m/z = 568.34 (M + 1) |
| I.bf.001 | | Rt = 2.05 min; MS: m/z = 578.34 (M + 1) |
| I.bg.001 | | Rt = 1.57 min; MS: m/z = 572.02 (M + 1) |
| I.bh.001 | | Rt = 2.11 min; MS: m/z = 598.39 (M + 1) |
| I.bi.001 | | Rt = 1.88 min; MS: m/z = 578.34 (M + 1) |
| I.bj.001 | | Rt = 2.19 min; MS: m/z = 660.36 (M + 1) |
| I.bk.001 | | Rt = 2.02 min; MS: m/z = 600.35 (M + 1) |
| I.bm.001 | | Rt = 2.19 min; MS: m/z = 678.36 (M + 1) |
| I.bn.001 | | Rt = 2.21 min; MS: m/z = 674.36 (M + 1) |
| I.bo.001 | | Rt = 2.22 min; MS: m/z = 630.41 (M + 1) |
| I.bp.001 | | Rt = 2.08 min; MS: m/z = 622.33 (M + 1) |
| I.bq.001 | | Rt = 1.98 min; MS: m/z = 606.33 (M + 1) |
| I.br.001 | | Rt = 2.12 min; MS: m/z = 640.29 (M + 1) |
| I.bs.001 | | Rt = 1.97 min; MS: m/z = 618.37 (M + 1) |
| I.bt.001 | | Rt = 1.99 min; MS: m/z = 588.35 (M + 1) |
| I.bu.001 | | Rt = 1.96 min; MS: m/z = 588.35 (M + 1) |
| I.bv.001 | | Rt = 1.65 min; MS: m/z = 679.27 (M + 1) |
| I.by.001 | | Rt = 1.80 min; MS: m/z = 536.33 (M + 1) |
| I.bz.001 | | Rt = 1.74 min; MS: m/z = 550.33 (M + 1) |
| I.bx.001 | 115-120 | Rt = 1.95 min; MS: m/z = 496 (M − 1) |
| I.b.193 | | Rt = 1.93 min; MS: m/z = 558.58 (M + 1) |
| I.ca.001 | | Rt = 1.96 min; MS: m/z = 622 (M − 1) |
| I.cb.001 | | Rt = 1.88 min; MS: m/z = 584 (M + 1) |
| I.cc.001 | | Rt = 1.74 min; MS: m/z = 513 (M + 1) |
| I.cd.001 | | Rt = 1.01 min; MS: m/z = 582.00 (M + 1) |
| I.ce.001 | | Rt = 1.31 min; MS: m/z = 587.21 (M − 1) |
| I.cf.001 | | Rt = 2.12 min; MS: m/z = 567.59 (M + 1) |
| I.cg.001 | 179-182 | Rt = 2.01 min; MS: m/z = 594.62 (M + 1) |
| I.ch.001 | | Rt = 2.09 min; MS : m/z = 596.12 (M − 1) |
| I.ci.001 | | Rt = 1.96 min; MS: m/z = 540.56 (M + 1) |
| I.cj.001 | | Rt = 1.88 min; MS: m/z = 530.55 (M + 1) |
| I.ck.001 | 88-92 | Rt = 2.07 min; MS: m/z = 612.63 (M + 1) |
| I.cl.001 | | Rt = 1.97 min; MS: m/z = 626.65 (m + 1) |
| I.cm.001 | | Rt = 1.9 min; MS: m/z = 674.07 (M − 1) |
| I.cn.001 | | Rt = 1.84 min; MS: m/z = 611.21 (M − 1) |
| I.co.001 | | Rt = 2.04 min; MS: m/z = 616.59 (M + 1) |
| I.cp.001 | | Rt = 1.87 min; MS: m/z = 620.20 (M + 1) |
| I.cq.001 | | Rt = 2.00 min; MS: m/z = 629 (M + 1) |
| I.cr.001 | | Rt = 2.01 min; MS: m/z = 636.18 (M − 1) |
| I.cs.001 | | Rt = 1.99 min; MS: m/z = 598 (M + 1) |
| I.ct.001 | | Rt = 1.95 min; MS: m/z = 613 (M + 1) |
| I.cu.001 | | Rt = 2.04 min; MS: m/z = 668.69 (M + 1) |
| I.cv.001 | 70-72 | |
| I.cw.001 | | Rt = 2.07 min; MS: m/z = 554 (M + 1) |
| I.cx.001 | | Rt = 1.95 min; MS: m/z = 543 (M + 1) |
| I.cy.001 | | Rt = 1.01 min; MS: m/z = 582.00 (M + 1) |
| I.cz.001 | | Rt = 1.99 min; MS: m/z = 610.63 (M + 1) |
| I.da.001 | | Rt = 1.90 min; MS: m/z = 598.35 (M + 1) |
| I.db.001 | | Rt = 1.97 min; MS: m/z = 657.68 (M + 1) |
| I.dc.001 | | Rt = 1.90 min; MS: m/z = 616.35 (M + 1) |

The compounds according to the present invention can be prepared according to the above-mentioned reaction schemes, in which, unless otherwise stated, the definition of each variable is as defined above for a compound of formula (I).

### BIOLOGICAL EXAMPLES

### Phytophthora infestans/Tomato/Leaf Disc Preventative

### Tomato Late Blight

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

Compounds I.a.001, I.b.001, I.c.001, I.e.001, I.f.001, I.g.001, I.h.001, I.i.001, I.j.001, I.k.001, I.m.001, I.n.001, I.s.001, I.v.001, I.y.001, I.z.001, I.ac.001, I.ad.001, I.ae.001, I.af.001, I.ah.001, I.ai.001, I.aj.001, I.ak.001, I.am.001, I.an.001, I.ao.001, I.ap.001, I.aq.001, I.ar.001, I.as.001, I.at.001, I.au.001, I.av.001, I.aw.001, I.ax.001, I.ay.001, I.ba.001, I.bb.001, I.be.001, I.bf.001, I.bh.001, I.bi.001, I.bj.001, I.bk.001, I.bm.001, I.bo.001, I.bp.001, I.bq.001, I.br.001, I.bs.001, I.bu.001, I.by.001 and I.bz.001 at 200 ppm give at least 80% disease control in this test when compared to untreated control leaf disks under the same conditions, which show extensive disease development.

### Phytophthora infestans/Potato/Preventative

### Potato Late Blight

2-week old potato plants cv. Bintje are sprayed in a spray chamber with the formulated test compound diluted in water. The test plants are inoculated by spraying them with a sporangia suspension 2 days after application. The inoculated test plants are incubated at 18° C. with 14 h light/day and 100% rh in a growth chamber and the percentage leaf area covered by disease is assessed when an appropriate level of disease appears on untreated check plants (5-7 days after application).

Compounds I.a.001, I.b.001 and I.c.001 at 200 ppm give at least 80% disease control in this test when compared to untreated control leaf disks under the same conditions, which show extensive disease development.

### Phytophthora infestans/Potato/Long Lasting

### Potato Late Blight

2-week old potato plants cv. Bintje are sprayed in a spray chamber with the formulated test compound diluted in water. The test plants are inoculated by spraying them with a sporangia suspension 6 days after application. The inoculated test plants are incubated at 18° C. with 14 h light/day and 100% rh in a growth chamber and the percentage leaf area covered by disease is assessed when an appropriate level of disease appears on untreated check plants (9-11 days after application).

Compounds I.a.001, I.b.001 and I.c.001 at 200 ppm give at least 80% disease control in this test when compared to untreated control leaf disks under the same conditions, which show extensive disease development.

### Plasmopara viticola/Grape/Leaf Disc Preventative

### Grape Downy Mildew

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

Compounds I.a.001, I.b.001, I.c.001, I.e.001, I.f.001, I.g.001, I.h.001, I.i.001, I.j.001, I.k.001, I.m.001, I.n.001, I.s.001, I.v.001, I.y.001, I.z.001, I.ab.001, I.ac.001, I.ad.001, I.ae.001, I.af.001, I.ah.001, I.ai.001, I.aj.001, I.ak.001, I.am.001, I.an.001, I.ao.001, I.ap.001, I.aq.001, I.ar.001, I.as.001, I.at.001, I.au.001, I.av.001, I.ax.001, I.ay.001, I.ba.001, I.bb.001, I.bc.001, I.be.001, I.bf.001, I.bh.001, I.bi.001, I.bj.001, I.bk.001, I.bm.001, I.bp.001, I.bq.001, I.br.001, I.bs.001, I.bt.001, I.bu.001, I.by.001 and I.bz.001 at 200 ppm give at least 80% disease control in this test when compared to untreated control leaf disks under the same conditions, which show extensive disease development.

### Plasmopara viticola/Grape/Preventative

### Grape Downy Mildew

5-week old grape seedlings cv. Gutedel are sprayed in a spray chamber with the formulated test compound diluted in water. The test plants plants are inoculated by spraying a sporangia suspension on their lower leaf surface one day after application. The inoculated test plants are incubated at 22° C. and 100% rh in a greenhouse and the percentage leaf area covered by disease is assessed when an appropriate level of disease appears on untreated check plants (6-8 days after application).

Compounds I.a.001, I.b.001 and I.c.001 at 200 ppm give at least 80% disease control in this test when compared to untreated control leaf disks under the same conditions, which show extensive disease development.

### Plasmopara viticola/Grape/Long Lasting

### Grape Downy Mildew

5-week old grape seedlings cv. Gutedel are sprayed in a spray chamber with the formulated test compound diluted in water. The test plants are inoculated by spraying a sporangia suspension on their lower leaf surface 6 days after application. The inoculated test plants are incubated at 22° C. and 100% rh in a greenhouse and the percentage leaf area covered by disease is assessed when an appropriate level of disease appears on untreated check plants (11-13 days after application).

Compounds I.a.001, I.b.001 and I.c.001 at 200 ppm give at least 80% disease control in this test when compared to untreated control leaf disks under the same conditions, which show extensive disease development.

### Pythium ultimum/Liquid Culture

### Seedling Damping Off

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added. The test plates are incubated at 24° C. and the inhibition of growth is determined photometrically 2-3 days after application.

Compounds I.a.001, I.b.001, I.c.001, I.e.001, I.f.001, I.g.001, I.i.001, I.j.001, I.k.001, I.m.001, I.n.001, I.y.001, I.z.001, I.ac.001, I.ad.001, I.ai.001, I.an.001, I.ap.001, I.aq.001, I.ar.001, I.as.011, I.av.001, I.aw.001, I.ax.001, I.bb.001, I.bc.001, I.be.001, I.bh.001, I.bi.001, I.bk.001, I.bp.001, I.bq.001, I.bu.001, I.by.001 and I.bz.001 at 200 ppm give at least 80% disease control in this test when compared to untreated control leaf disks under the same conditions, which show extensive disease development.

1. A compound of formula I: 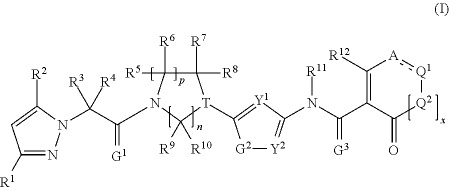
wherein
G¹, G² and G³ are independently O or S;
T is CR¹³ or N;
Y¹ and Y² are independently CR¹⁴ or N;
A is C(R¹⁵R¹⁶), C(═O), C(═S), NR²¹, O or S;
Q¹ is C(R¹⁷R¹⁸), C(═O), C(═S), NR²¹, O or S;
Q² is C(R¹⁹R²⁰), C(═O), C(═S), NR²¹, O or S;
the bond between A and Q¹ is a single bond or a double bond;
n is 1 or 2;
p is 1 or 2, providing that when n is 2, p is 1;
x is 0 or 1, providing that when x is 1, Q¹ and Q² cannot both be oxygen;
R¹, R², R³, R⁴, R⁵, R⁶, R⁷, R⁸, R⁹, R¹⁰, R¹³ and R¹⁴ each independently are hydrogen, halogen, cyano, C₁-C₄alkyl, C₃-C₅cycloalkyl or C₁-C₄haloalkyl;
R¹¹ is hydrogen, C₁-C₄alkyl, C₃-C₅cycloalkyl or C₁-C₄alkoxy;
R¹² is hydroxyl, O⁻M⁺, OC(═O)R²⁵, amino or NHR²²;
M⁺ is a metal cation or ammonium cation,
R¹⁵, R¹⁶R¹⁷, R¹⁸, R¹⁹ and R²⁰ each independently are hydrogen, halogen, hydroxyl, amino, cyano, C₁-C₈alkyl, C₁-C₈alkylcarbonyl, C₂-C₈alkenyl, C₂-C₈alkynyl, C₃-C₈cycloalkyl, C₁-C₈alkoxy, C₁-C₈alkoxycarbonyl, C₁-C₈alkylthio, C₁-C₈alkylsulfonyl, C₁-C₈alkylsulfinyl, aryl, arylcarbonyl, heteroaryl or NHR²², wherein alkyl, alkenyl, alkynyl, cycloalkyl alkoxy, aryl and heteroaryl are optionally substituted by one or more R²³; and wherein
R¹⁵ and R¹⁶, R¹⁷ and R¹⁸, and/or R¹⁹ and R²⁰ may together form a saturated three- to six-membered alicyclic or heterocyclic ring wherein the aliyclic and heterocyclic rings are optionally substituted by one or more R²⁴; and/or
R¹⁵ and R¹⁷, and/or R¹⁸ and R¹⁹ together form a saturated or partially unsaturated four- to seven-membered alicyclic or heterocyclic ring wherein the aliyclic and heterocyclic rings are optionally substituted by one or more R²⁴; and/or
R¹⁵ and R¹⁹ together form a saturated or partially unsaturated four- to seven-membered alicyclic or heterocyclic ring wherein the aliyclic and heterocyclic rings are optionally substituted by one or more R²⁴;
R²¹ and R²² each independently are hydrogen, C₁-C₈alkyl, C₁-C₈haloalkyl C₂-C₈alkenyl, C₁-C₈haloalkenyl C₂-C₈alkynyl, C₂-C₈haloalkynyl, C₃-C₈cycloalkyl, C₃-C₈halocycloalkyl, C₁-C₈alkoxy, C₁-C₈haloalkoxy, C₁-C₈alkylcarbonyl, C₁-C₈alkoxycarbonyl, C₁-C₈haloalkylcarbonyl, C₁-C₈alkylsulfonyl, C₁-C₈haloalkylsulfonyl, amino, NH(C₁-C₈alkyl), N(C₁-C₈alkyl)₂, aryl or heterocycyl, wherein aryl and heterocyclyl are optionally substituted by one or more R²⁴;
each R²³ independently is halogen, cyano, amino, nitro, hydroxyl, mercapto, C₁-C₈alkyl, C₂-C₈alkenyl, C₂-C₈alkynyl, C₃-C₈cycloalkyl, C₃-C₈cycloalkyl-C₁-C₄alkyl, C₃-C₈cycloalkyl-C₁-C₄alkyloxy, C₃-C₈cycloalkyl-C₁-C₄alkylthio, C₁-C₈alkoxy, C₃-C₈cycloalkyloxy, C₁-C₈alkenyloxy, C₂-C₈alkynyloxy, C₁-C₈alkylthio, C₁-C₈alkylsulfonyl, C₁-C₈alkylsulfinyl, C₃-C₈cycloalkylthio, C₃-C₈cycloalkylsulfonyl, C₃-C₈cycloalkylsulfinyl, aryl, aryloxy, arylthio, arylsulfonyl, arylsulfinyl, aryl-C₁-C₄alkyl, aryl-C₁-C₄alkyloxy, aryl-C₁-C₄alkylthio, heterocyclyl, heterocycyl-C₁-C₄alkyl, heterocycyl-C₁-C₄alkyloxy, heterocyclyl-C₁-C₄alkylthio, NH(C₁-C₈alkyl), N(C₁-C₈alkyl)₂, C₁-C₄alkylcarbonyl, C₃-C₈cycloalkylcarbonyl, C₂-C₈alkenylcarbonyl, C₂-C₈alkynylcarbonyl, wherein alkyl, alkenyl, alkynyl, cycloalkyl, alkoxy, alkenyloxy, alkynyloxy and cycloalkoxy are optionally substituted by halogen, and wherein aryl and heterocyclyl are optionally substituted by one or more R²⁴;
each R²⁴ independently is halogen, cyano, C₁-C₄alkyl, C₁-C₄haloalkyl, C₁-C₄alkoxy or C₁-C₄haloalkoxy; and
R²⁵ is C₁-C₆alkyl or C₁-C₆alkoxy; or a salt or a N-oxide thereof.

2. A compound according to claim 1, wherein when x is 1, A is C(R¹⁵R¹⁶), NR²¹, O or S; Q¹ is C(R¹⁷R¹⁸), C(═O), C(═S), NR²¹, O or S; and Q² is C(R¹⁹R²⁰), NR²¹, O or S; and when x is 0 A is C(R¹⁵R¹⁶), C(═O), C(═S), NR²¹, O or S; and Q¹ is C(R¹⁷R¹⁸), NR²¹, O or S.

3. A compound according to claim 1, wherein no more than two of A, Q¹ and Q² are NR²¹, O or S.

4. A compound according to claim 1, wherein the compound of formula I is a compound of formula I.d 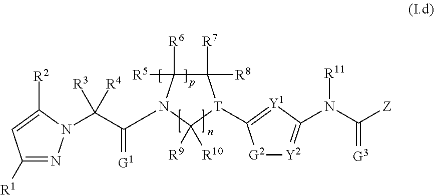
wherein Z is selected from Z1 to Z19 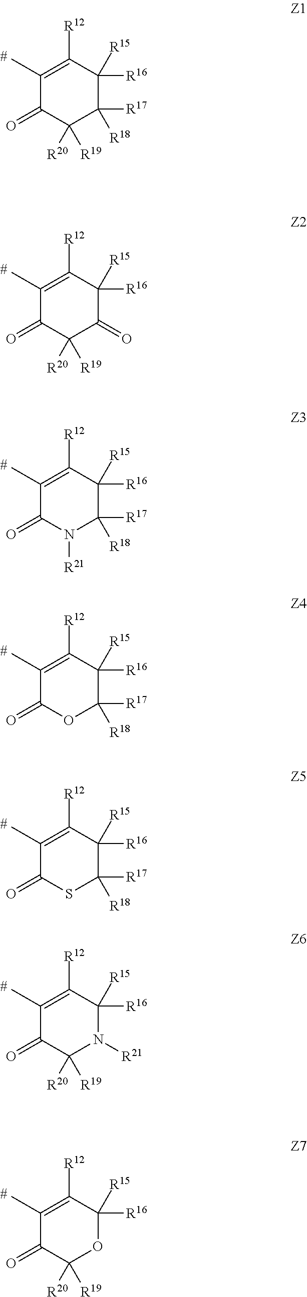 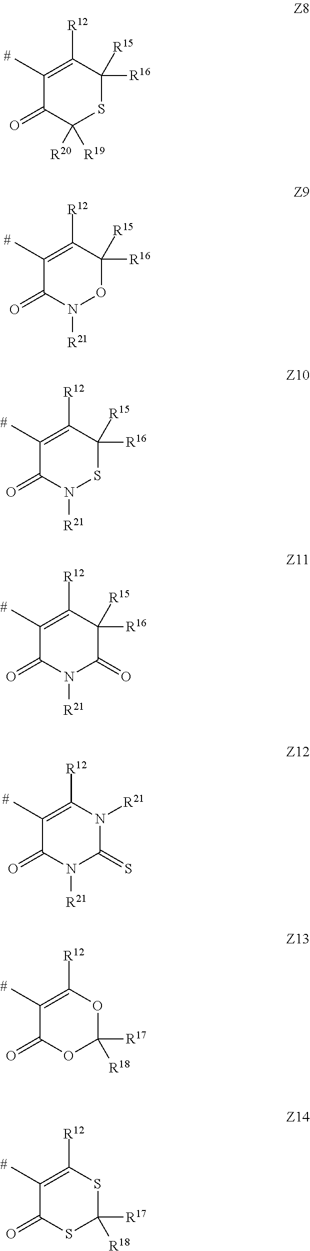 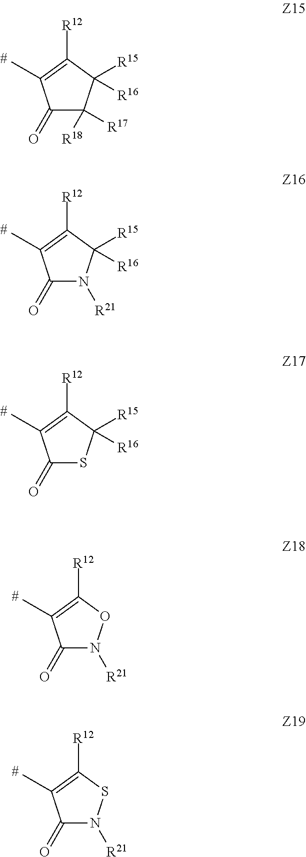
and G¹, G², G³, T, Y¹, Y², n, p, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰ and R²¹ are as defined for the compound of formula I in claim 1.

5. A compound according to claim 4, wherein Z is selected from Z1, Z2, Z3, Z4, Z7, Z8, Z9, Z11, Z12, Z13, Z16, Z17 and Z18.

6. A compound according to claim 1, wherein R¹² is hydroxyl or O⁻M⁺.

7. A compound according to claim 1, wherein R¹ and R² are independently methyl or halomethyl.

8. A compound according to claim 1, wherein R³, R⁴, R⁵, R⁶, R⁷, R⁸, R⁹, R¹⁰, R¹³ and R¹⁴ are independently hydrogen, halogen, methyl or halomethyl.

9. A compound according to claim 1, wherein R¹¹ is hydrogen or methyl.

10. A compound according to claim 1, wherein G¹ and G³ are O.

11. A compound according to claim 1, wherein p is 1 and n is 2.

12. A compound of formula II.b 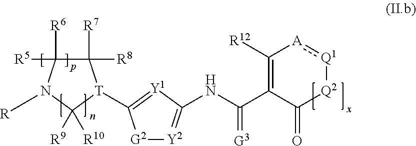
wherein R is hydrogen or a protecting group and G², G³, T, Y¹, Y², n, p, R⁵, R⁶, R⁷, R⁸, R⁹, R¹⁰, R¹², A, Q¹, Q² and x are as defined for a compound of formula I in claim 1 or a salt or N-oxide thereof, or
a compound of formula III 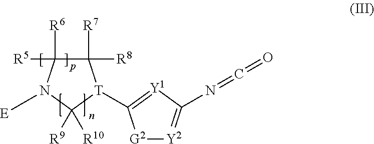
wherein E is hydrogen, a protecting group or group M 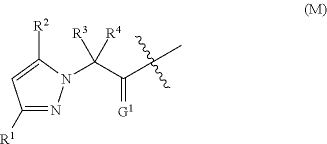
and G¹, G², T, Y¹, Y², n, p, R¹, R², R³, R⁴, R⁵, R⁶, R⁷, R⁸, R⁹ and R¹⁰ are as defined for a compound of formula I in claim 1 or a salt or N-oxide thereof, or
a compound of formula IV 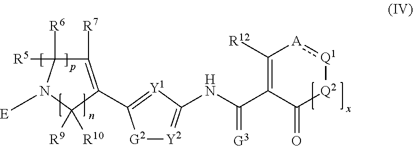
wherein E is hydrogen, a protecting group or group M 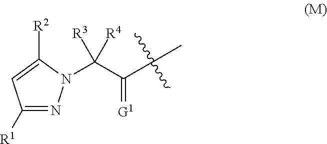
and G¹, G², G³, Y¹, Y², n, p, R¹, R², R³, R⁴, R⁵, R⁶, R⁷, R⁹, R¹⁰, R¹², A, Q¹, Q² and x are as defined for a compound of formula I in claim 1 or a salt or N-oxide thereof, or
a compound of formula V 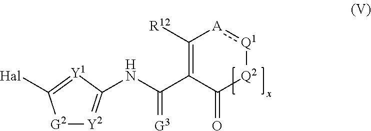
wherein Hal is halogen and G², G³, Y¹, Y², R¹², A, Q¹, Q² and x are as defined for a compound of formula I in claim 1 or a salt or N-oxide thereof.

13. A composition comprising at least one compound as defined in claim 1 and an agriculturally acceptable carrier, optionally comprising an adjuvant, and optionally comprising one or more additional pesticidally active compounds.

14. A method of controlling or preventing an infestation of plants, propagation material thereof, harvested crops or non-living materials by phytopathogenic or spoilage microorganisms or organisms potentially harmful to man, which comprises the application of a compound as defined in claim 1, to the plant, to parts of the plants or to the locus thereof, to propagation material thereof or to any part of the non-living materials.
